(19) United States
(12) Patent Application Publication
(10) Pub. No.: US 2022/0354929 A1
(43) Pub. Date: Nov. 10, 2022

(54) PEPTIDES AND USES THEREOF

(71) Applicant: LATERAL IP PTY LTD, Melbourne, Victoria (AU)

(72) Inventor: Andrew Gearing, Melbourne, Victoria (AU)

(73) Assignee: LATERAL IP PTY LTD, Melbourne, Victoria (AU)

(21) Appl. No.: 17/622,202

(22) PCT Filed: Jul. 8, 2020
(86) PCT No.: PCT/AU2020/050713

§ 371 Date: Dec. 22, 2021

(30) Foreign Application Priority Data
Jul. 9, 2019 (AU) 2019902437

(51) Int. Cl. A61K 38/22; A61P 25/02; A61P 29/00

(52) U.S. Cl. CPC A61K 38/2257; A61P 25/02; A61P 29/00

(57) ABSTRACT

Disclosed herein are methods and compositions for treating or preventing neuropathic pain in a subject, the method comprising administering to a subject a therapeutically effective amount of prolactin, or a functional variant thereof, wherein the functional variant comprises a peptide of formula (I) or a pharmaceutically acceptable salt thereof: R¹-C-R-I-X₁-X₂-X₃-X₄-N-C-R² (I) (SEQ ID NO:1) wherein X₁ is an amino acid residue selected from isoleucine (I) and valine (V); X₂ is an amino acid residue selected from histidine (H) and tyrosine (Y); X₃ is an amino acid residue selected from aspartic acid (D) and asparagine (N); X₄ is an amino acid Nresidue selected from asparagine (N) and serine (S); R¹ is selected from the group consisting of YLKLLK, LKLLK, KLLK, LLK, LL, K or R¹ is absent; and R² is G (glycine), or R² is absent.

### FIELD OF THE INVENTION

The invention relates generally to peptides and compositions useful in treating or preventing neuropathic pain, and methods of their use.

### BACKGROUND

All references, including any patent or patent application cited in this specification are hereby incorporated by reference to enable full understanding of the invention. Nevertheless, such references are not to be read as constituting an admission that any of these documents forms part of the common general knowledge in the art, in Australia or in any other country.

Pain can be a debilitating sensory experience that is typically associated with tissue damage and/or an underlying neurological disorder. Pain, whether acute or chronic, may arise in the absence of any detectable stimulus, injury or underlying disease. Acute pain usually lasts for short periods (e.g., a few hours or days), and will typically disappear upon cessation of the underlying stimulus. By contrast, chronic pain lasts for longer periods (e.g., weeks or months), and can persist even in the absence of an underlying stimulus.

There are two widely recognized types of pain—nociceptive pain and neuropathic pain. Nociceptive pain is the result of potentially harmful stimulation of the sensory nerve fibres, detected by nociceptors around the body that respond to mechanical or physical damage. Nociceptive pain serves a protective biological function by warning of tissue damage, to cause withdrawal from the noxious stimulus. Nociceptive pain may result from thermal damage, such as burns or frostbite, or result from mechanical trauma such as laceration or pressure.

In contrast to nociceptive pain, neuropathic pain is caused by a primary lesion, malfunction or dysfunction in the peripheral or central nervous system. Neuropathic pain has no protective effect and can develop days or months after an injury or after resolution of a disease state, and is frequently long-lasting and chronic.

Neuropathic pain may result from nerve damage caused by a trauma such as a sporting injury, an accident, a fall or a penetrating injury or the nerve damage may result from a disease process such as stroke, viral infections, exposure to toxins, degenerative diseases and diabetes. The prevalence of disease states which may result in the development of neuropathic pain conditions, such as diabetic neuropathy and post-herpetic neuralgia, is increasing and therefore an increasing number of people are suffering chronic neuropathic pain symptoms.

Although there are effective remedies for treating nociceptive pain, neuropathic pain is often resistant to available analgesic drugs. In addition, current therapies such as tricyclic antidepressants, anticonvulsants, opioid and non-opioid analgesics have significant side effects such as sedation and sleepiness and in the case of opioid analgesics, the risk of drug tolerance and drug dependency or addiction. Moreover, there are currently few useful options available for the treatment of neuropathic pain in the absence of an analgesic effect on nociceptive pain. Accordingly, there remains an urgent need for new and alternative options that are effective for the selective treatment of neuropathic pain, with limited or no side effects. The present invention solves, or partly alleviates, this problem by providing compounds that are effective at alleviating neuropathic pain with minimal or no analgesic effect on nociceptive pain.

### SUMMARY OF THE INVENTION

In an aspect disclosed herein, there is provided a method of treating or preventing neuropathic pain in a subject, the method comprising administering to a subject a therapeutically effective amount of prolactin, or a functional variant thereof, wherein the functional variant comprises a peptide of formula (I):

(SEQ ID NO: 1)
R¹-C-R-I-X₁-X₂-X₃-X₄-N-C-R² (I)

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent, or R² is a pharmaceutically acceptabR¹ carrier.

In an embodiment, the peptide of formula (I) is selected from the group consisting of amino acid sequence CRIIHNNNC (SEQ ID NO:2), CRIIHNNNCG (SEQ ID NO:3), CRIVYDSNC (SEQ ID NO:4) and CRIVYDSNCG (SEQ ID NO:5).

In an embodiment, said therapeutically effective amount alleviates neuropathic pain in the subject in the absence of a therapeutically effective analgesic effect on nociceptive pain.

In an embodiment, the subject is a human.

In an embodiment, the subject is selected from the group consisting of a feline, a canine and an equine.

In an embodiment, the neuropathic pain is associated with a condition selected from the group consisting of diabetic neuropathy; Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HIV sensory neuropathy; hereditary motor and sensory neuropathy (HMSN); hereditary sensory neuropathy (HSN); hereditary sensory and autonomic neuropathy; hereditary neuropathy with ulcero-mutilation; nitrofurantoin neuropathy; tomaculous neuropathy; neuropathy caused by nutritional deficiency, neuropathy caused by kidney failure, trigeminal neuropathic pain, atypical odontalgia (phantom tooth pain), burning mouth syndrome, complex regional pain syndrome, repetitive strain injury, drug-induced peripheral neuropathy and peripheral neuropathy associated with infection.

In an embodiment, the method further comprises administering to the subject a therapeutically effective amount of a second agent capable of alleviating pain in the subject, wherein the second agent is not prolactin, or a functional variant thereof, as herein described.

In another aspect disclosed herein, there is provided a pharmaceutical composition comprising prolactin, or a functional variant thereof, for the treatment and prevention of neuropathic pain in a subject, wherein the functional variant comprises a peptide of formula (I):

(SEQ ID NO: 1)
R¹-C-R-I-X₁-X₂-X₃-X₄-N-C-R² (I)

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent or R² is a pharmaceutically acceptable carrier.

In another aspect disclosed herein, there is provided use of prolactin, or a functional variant thereof, in the manufacture of a medicament for the treatment and prevention of neuropathic pain in a subject, wherein the functional variant comprises a peptide of formula (I):

(SEQ ID NO: 1)
R¹-C-R-I-X₁-X₂-X₃-X₄-N-C-R² (I)

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent.

In another aspect disclosed herein, there is provided use of a pharmaceutical composition comprising (i) prolactin, or a functional variant thereof, wherein the functional variant comprises a peptide of formula (I),

(SEQ ID NO: 1)
R¹-C-R-I-X₁-X₂-X₃-X₄-N-C-R² (I)

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent; and
(ii) a second agent capable of alleviating pain in the subject, wherein the second agent is not prolactin, or a functional variant thereof, as herein described.

In another aspect disclosed herein, there is provided an analgesic composition comprising prolactin, or a functional variant thereof, wherein the functional variant comprises a peptide of formula (I):

(SEQ ID NO: 1)
R¹-C-R-I-X₁-X₂-X₃-X₄-N-C-R² (I)

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent.

In another aspect disclosed herein, there is provided an analgesic composition comprising a therapeutically effective amount of a peptide, or a pharmaceutically acceptable salt thereof, wherein the peptide consists of amino acid sequence CRIIHNNNC (SEQ ID NO:2), CRIIHNNNCG (SEQ ID NO:3), CRIVYDSNC (SEQ ID NO:4) and CRIVYDSNCG (SEQ ID NO:5).

In another aspect disclosed herein, there is provided a pharmaceutical composition comprising prolactin, or a functional variant thereof, wherein the functional variant comprises a peptide of formula (I):

(SEQ ID NO: 1)
R¹-C-R-I-X₁-X₂-X₃-X₄-N-C-R² (I)

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent or R² is a pharmaceutically acceptable carrier.

In an embodiment, the peptide is selected from the group consisting of

(SEQ ID NO: 6)
YLKLLKCRIIHNNNC,
(SEQ ID NO: 7)
LKLLKCRIIHNNNC,
(SEQ ID NO: 8)
KLLKCRIIHNNNC,
(SEQ ID NO: 9)
LLKCRIIHNNNC,
(SEQ ID NO: 10)
LKCRIIHNNNC,
(SEQ ID NO: 11)
KCRIIHNNNC,
(SEQ ID NO: 12)
YLKLLKCRIIHNNNCG,
(SEQ ID NO: 13)
LKLLKCRIIHNNNCG,
(SEQ ID NO: 14)
KLLKCRIIHNNNCG,
(SEQ ID NO: 15)
LLKCRIIHNNNCG,
(SEQ ID NO: 16)
LKCRIIHNNNCG
and
(SEQ ID NO: 17)
KCRIIHNNNCG.

In another aspect disclosed herein, there is provided a composition comprising a therapeutically effective amount of a peptide, or a pharmaceutically acceptable salt thereof, wherein the peptide consists of amino acid sequence

(SEQ ID NO: 2)
CRIIHNNNC,
(SEQ ID NO: 3)
CRIIHNNNCG,
(SEQ ID NO: 4)
CRIVYDSNC
and
(SEQ ID NO: 5)
CRIVYDSNCG.

### BRIEF DESCRIPTION OF THE FIGURES

FIG. 1 is a schematic diagram showing the preparation of spinal cord slices and whole cell recording sites in Chung models of neuropathic pain.

FIG. 2 shows summary data of the changes in membrane potential and neuroneal input resistance associated with peptide SEQ ID NO:2 (also referred to herein as “LAT7771”)-induced responses in dorsal horn neurones compared to control resting state, from Chung models of neuropathic pain. (N=11, *P<0.05, Student's paired t-test)

FIG. 3 shows summary data of the changes in membrane potential and neuronal input resistance associated with LAT7771-induced responses in dorsal horn neurones compared to control resting state, from Chung models of neuropathic pain. (N=11)

FIG. 4 shows LAT7771-induced membrane depolarisation and an increase in spontaneous firing. A and B show samples of a continuous record of a spontaneously active cell in the absence (A) and presence (B) of LAT7771. C: Voltage-current (VI) relations of the same cell as shown in A and B. Voltage responses to current injection are shown superimposed. D and E show plots of the data shown in C with amplitude of voltage responses measured at the peak (D) of the response and at steady-state (E; end of the response). Note the almost parallel shift in the slope indicating little change in conductance.

FIG. 5 shows LAT7771 induced membrane depolarisation and enhanced inward rectification in a dorsal horn neurone from a Chung model of neuropathic pain. A and B: Voltage-current (VI) relations of the cell in the absence (A) and presence (B) of LAT7771. Voltage responses to current injection are shown superimposed. C and D shows a plot of the data shown in A and B. Note the almost parallel shift in the slope indicating little change in conductance at depolarised potentials but reduction in the slope at membrane potentials more negative than around −70 mV, the latter indicating enhanced inward rectification.

FIG. 6 shows LAT7771 enhanced inhibitory synaptic transmission in a dorsal horn neuronee from a Chung model of neuropathic pain with little effect on postsynaptic membrane properties. A. Samples of a continuous record showing LAT7771 induced a minimal membrane depolarisation in this cell. B and C: Voltage-current (VI) relations of the cell shown in A in the absence (B) and presence (C) of LAT7771. Voltage responses to current injection are shown superimposed. Note little change observed in the VI relations. D. Same neurone as A, B and C showing superimposed inhibitory postsynaptic potentials (IPSPs) evoked by electrical stimulation of dorsal root afferents (0.1 Hz) in the absence (black) and presence (red) of LAT7771. E shows superimposed averages of the data shown in D. Note the increase in peak amplitude of IPSPs in the presence of LAT7771.

FIG. 7 shows that LAT7771 and LAT8881 (human growth hormone derived analgesic compound having the amino acid sequence YLRIVQCRSVEGSCGF) enhanced inhibitory synaptic transmission in a dorsal horn neurone from a Chung model of neuropathic pain with little effect on postsynaptic membrane properties. A. Samples of a continuous record showing VI relations in control (black), in LAT7771 (red), following wash (blue) and subsequently in LAT8881 (green). LAT7771 (red) and LAT8881 (green) had little or no effect on postsynaptic properties in this cell. B. Same neurone as A showing superimposed postsynaptic potentials evoked by electrical stimulation of dorsal roots (0.1 Hz). In control (black), excitatory postsynaptic potentials (EPSPs) were predominantly evoked by stimulation of dorsal roots. In the presence of LAT7771, EPSPs were suppressed and inhibitory postsynaptic potentials revealed (red), an effect partly reversible following washout of LAT7771 (blue). Subsequent application of LAT8881, similar to LAT7771, again enhanced inhibitory synaptic transmission whilst suppressing EPSPs. C. Superimposed averages of the responses shown in B. Note the presence of significant IPSPs in the presence of LAT7771 (red) and LAT8881 (green) compared to control (black) and post-LAT7771 wash.

FIG. 8 shows LAT7771 enhanced inhibitory synaptic transmission in a dorsal horn neurone from a Chung model of neuropathic pain A. Samples of a continuous record showing superimposed post synaptic potentials evoked in response to electrical stimulation of dorsal roots. In the absence of LAT7771, excitatory postsynaptic potentials (EPSPs) were predominant (control 1 and control 2). Subsequently in the presence of LAT7771, EPSPs were reduced and inhibitory postsynaptic potentials (IPSPs) dominated the response. B shows superimposed averages of the responses shown in A. Note the progressive suppression of EPSPs and appearance of IPSPs in the presence of LAT7771. C. Same neurone as A and B showing a time-course plot of postsynaptic responses in control and subsequently in the presence of LAT7771. Note the suppression of EPSPs and dominance by IPSPs in the presence of LAT7771.

FIG. 9 shows summary data showing changes in membrane potential and neuronal input resistance associated with peptide SEQ ID NO:3 (also referred to herein as “LAT7772”)-induced responses in dorsal horn neurones from Chung models of neuropathic pain. (n=5, *P<0.05 vs rest, Student's paired t-test).

FIG. 10 Summary data showing changes in membrane potential and neuronal input resistance associated with LAT7772-induced responses in dorsal horn neurones from Chung models of neuropathic pain, (n=5).

FIG. 11 shows A: LAT7772 induced hyperpolarisation associated with a reduction in neuronal input resistance. B: Voltage-current (VI) relations of the same cell as shown in A. Voltage responses to current injection are shown superimposed. C shows a plot of the data from the same cell as B in the presence of LAT7772. Note the reduction in the slope indicating a reduction in neuronal input resistance indicating ion channel opening. The plots intersect around −60 mV (approaching the reversal potential for chloride ions under our recording conditions). D. LAT7772 also suppressed dorsal root afferent-mediated excitatory synaptic transmission. Samples of a continuous record showing superimposed electrically-evoked (0.1 Hz) EPSPs in the absence (1, black) and presence (2, red) of LAT7772 with the averages of these records shown superimposed below. Note the suppression of dorsal root stimulation-evoked EPSPs in LAT7772.

FIG. 12 shows LAT7772 induced hyperpolarisation associated with a reduction in neuronal input resistance B: Voltage-current (VI) relations of the same cell as shown in A. Voltage responses to current injection are shown superimposed. C shows a plot of the data from the same cell as B in the presence of LAT7772. Note the reduction in the slope indicating a reduction in neuronal input resistance indicating ion channel opening. The plots intersect around −78 mV (midway between the reversal potentials for chloride and potassium ions under our recording conditions). D. LAT7772 also suppressed dorsal root afferent-mediated excitatory synaptic transmission. Samples of a continuous record showing superimposed electrically-evoked (0.1 Hz) EPSPs in the absence (1, black) and presence (2, red) of LAT7772 with the averages of these records shown superimposed below. Note the suppression of dorsal root stimulation-evoked EPSPs in LAT7772.

FIG. 13 shows LAT7772 induced depolarisation associated with a reduction in neuronal input resistance A: Voltage-current (VI) relations are shown with voltage responses to current injection superimposed. B: plot of the data shown in A. Note the reduction in the slope indicating a reduction in neuronal input resistance indicating ion channel opening. The plots intersect around −60 mV (close to the reversal potential for chloride ions under our recording conditions). C. LAT7772 also suppressed dorsal root afferent-mediated excitatory synaptic transmission. Samples of a continuous record showing superimposed electrically-evoked (0.1 Hz) EPSPs in the absence (1, black) and presence (2, red) of LAT7772 with the averages of these records shown superimposed below in D. Note the suppression of dorsal root stimulation-evoked late component EPSPs in LAT7772.

FIG. 14 shows summary data showing changes in membrane potential and neuronal input resistance associated with peptide SEQ ID NO:4 (also referred to herein as “LAT7773”)-induced responses in dorsal horn neurones from Chung models of neuropathic pain. (n=9, *P<0.05 vs rest, Student's paired t-test).

FIG. 15 shows summary data showing changes in membrane potential and neuronal input resistance associated with LAT7773-induced responses in dorsal horn neurones from Chung models of neuropathic pain, (n=9).

FIG. 16 shows LAT7773 induced little change in membrane potential whilst enhancing inward rectification and reducing dorsal root afferent-mediated synaptic inputs in this neurone. A: Voltage-current (VI) relations showing voltage responses to current injection superimposed. B shows a plot of the data from the same cell as A in the presence of LAT7773. Note the lack of change in conductance around the resting potential of −60 mV but the decrease in slope of the plot at more negative membrane potentials in LAT7773 indicating enhanced inward rectification in the presence of the compound. C and D. Same neurone as A and B showing superimposed EPSPs evoked by stimulation of the dorsal roots. Note the increase in the number of failures of stimulation in the presence of LAT7773 (red, D) compared to control (black, C). E shows the superimposed averages of the data shown in C and D.

FIG. 17 shows LAT7773 induced depolarisation associated with an increase in neuronal input resistance A: Voltage-current (VI) relations are shown with voltage responses to current injection superimposed. B: Plot of the data shown in A. Note the increase in the slope indicating an increase in neuronal input resistance consistent with ion channels closing. The plots intersect around −92 mV (close to the reversal potential for potassium ions under our recording conditions). C. LAT7773 had little effect on dorsal root afferent-mediated excitatory synaptic transmission. Samples of a continuous record showing superimposed electrically-evoked (0.1 Hz) EPSPs in the absence (1, black) and presence (2, red) of LAT7773.

FIG. 18 shows LAT7773 induced an increase in dorsal root afferent-mediated synaptic transmission with little effect on postsynaptic membrane properties. A: Voltage-current (VI) relations are shown with voltage responses to current injection superimposed. B: plot of the data shown in A. Note the decrease in the slope at negative membrane potentials in LAT7773 suggesting enhanced inward rectification with little effect on input resistance around resting potential. C, D and E: samples of a continuous record showing dorsal root afferent-mediated EPSPs and IPSPs were enhanced in the presence of LAT7773. D and E show EPSPs (D) and IPSPs (E) separated from the continuous record shown in C. Note both EPSPs and IPSPs were enhanced in LAT7773.

FIG. 19 shows summary data showing changes in membrane potential and neuronal input resistance associated with peptide SEQ ID NO:5 (also referred to herein as “LAT7774”)-induced responses in dorsal horn neurones from Chung models of neuropathic pain. (n=7).

FIG. 20 shows summary data showing changes in membrane potential and neuronal input resistance associated with LAT7774-induced responses in dorsal horn neurones from Chung models of neuropathic pain, (n=7).

FIG. 21 shows LAT7774 induced hyperpolarisation associated with a reduction in neuronal input resistance and activation of a potassium conductance A: Voltage-current (VI) relations in the absence (control, black) and presence (red) of LAT7774 showing voltage responses to current injection superimposed. B shows a plot of the data from the same cell as A in the absence and presence of LAT7774. Note the reduction in the slope indicating a reduction in neuronal input resistance indicating ion channel opening. The plots are projected to intersect around −95 mV (close to the reversal potential for potassium ions under our recording conditions). C. Same neurone showing the effect of LAT7774 on dorsal root afferent-mediated synaptic inputs. Samples of a continuous record showing superimposed EPSPs evoked by stimulation of the dorsal roots. Bottom traces show the averages of 1 and 2 superimposed. Note the small decrease in EPSPs in the presence of LAT7774.

FIG. 22 shows LAT7774 induced hyperpolarisation associated with a reduction in neuronal input resistance and activation of a chloride conductance A: Voltage-current (VI) relations in the absence (control, black) and presence (red) of LAT7774 showing voltage responses to current injection superimposed. B shows a plot of the data from the same cell as A in the absence and presence of LAT7774. Note the reduction in the slope indicating a reduction in neuronal input resistance and ion channel opening. The plots are projected to intersect around −55 mV (close to the reversal potential for chloride ions under our recording conditions). C. Same neurone showing the effect of LAT7774 on dorsal root afferent-mediated synaptic inputs. Samples of a continuous record showing superimposed EPSPs evoked by stimulation of the dorsal roots. Bottom traces show the averages of 1 and 2 superimposed. Note the lack of effect of LAT7774 on these potentials.

FIG. 23 shows LAT7774 induced depolarisation associated with an increase in neuronal input resistance and block of a potassium conductance, most likely an inwardly rectifying potassium conductance A: Voltage-current (VI) relations in the absence (control, black) and presence (red) of LAT7774 showing voltage responses to current injection superimposed. B shows a plot of the data from the same cell as A in the absence and presence of LAT7774. Note the increase in the slope indicating an increase in neuronal input resistance and ion channels closing. The plots are projected to intersect around −80 mV (approaching the reversal potential for potassium ions under our recording conditions). Note also the change in slope at potentials more negative than around −80 mV suggesting a decrease or change in voltage-dependence of inwardly rectifying potassium conductances. C. Same neurone showing the effect of LAT7774 on dorsal root afferent-mediated synaptic inputs. Samples of a continuous record showing superimposed EPSPs evoked by stimulation of the dorsal roots. Bottom traces show the averages of 1 and 2 superimposed. Note the lack of effect of LAT7774 on these potentials.

FIG. 24 shows LAT7774 induced depolarisation associated with an increase in neuronal input resistance and block of a potassium conductance. A: Voltage-current (VI) relations in the absence (control, black), presence (red) of LAT7774 and following wash of the compound (blue). Voltage responses to current injection are shown superimposed. B shows a plot of the data from the same cell as A in the absence and presence of LAT7774 and following washout of the compound. Note the increase in the slope indicating an increase in neuronal input resistance and ion channels closing. The plots are projected to intersect around −85 mV (approaching the reversal potential for potassium ions under our recording conditions). Note also the change in slope at potentials more negative than around −80 mV suggesting a decrease or change in voltage-dependence of inwardly rectifying potassium conductances. C. Same neurone showing the effect of LAT7774 on dorsal root afferent-mediated synaptic inputs. Samples of a continuous record showing superimposed EPSPs evoked by stimulation of the dorsal roots. Note the increase in EPSPs and increased tendency to reach threshold for firing in the presence of LAT7774. These effects of LAT7774 were at least partly reversible on washout of the drug.

FIG. 25 shows summary data showing changes in membrane potential and neuronal input resistance associated with prolactin-induced responses in dorsal horn neurones from Chung models of neuropathic pain. (n=3).

FIG. 26 shows summary data showing changes in membrane potential and neuronal input resistance associated with prolactin-induced responses in dorsal horn neurones from Chung models of neuropathic pain, (n=3).

FIG. 27 shows prolactin induced a marginal membrane depolarisation associated with an increase in neuronal input resistance. A. Membrane responses to depolarising current pulses shown superimposed in the absence (control, black) and presence (red) of prolactin. B: Voltage-current (VI) relations of the same cell as shown in A. Voltage responses to current injection are shown superimposed. C and D shows a plot of the data shown in B (measured at the peak of the response and at steady-state) in the absence and presence of prolactin. Note the increase in the slope indicating an increase in neuronal input resistance consistent with ion channels closing. The plots intersect around −90 mV (approaching the reversal potential for potassium ions under our recording conditions).

FIG. 28 shows prolactin had little effect on dorsal root stimulated inhibitory post-synaptic potentials (IPSPs). Samples of a continuous record showing superimposed IPSPs evoked by electrical stimulation of dorsal roots, at frequencies of 0.1 Hz (A) and 5 Hz (B), in the absence (control; black) and presence (red) of prolactin. Superimposed averages of these responses are shown far right. Note the lack of effect of LAT7771 on these IPSPs.

FIG. 29 shows summary data showing changes in normalised membrane potential associated with LAT7771, LAT7772, LAT7773, LAT7774 and prolactin-induced responses in dorsal horn neurones from Chung models of neuropathic pain.

FIG. 30 shows summary data showing changes in normalised membrane potential associated with LAT7771, LAT7772, LAT7773, LAT7774 and Prolactin-induced responses in dorsal horn neurones from Chung models of neuropathic pain.

FIG. 31 shows LAT7771 and LAT8881 (amino acid sequence YLRIVQCRSVEGSCGF) improved the outcome on neuropathic pain, as indicated by the reversal of mechanical allodynia in Chung model of neuropathic pain. Measurement of paw withdrawal threshold (PWT) was assessed once daily for three days before surgery (D-2, D-1 and D0) and once a week after surgery for monitoring the development of mechanical allodynia (D7). PWT was assessed before (BL) and 2 hours (2 hr) following drug or vehicle administration A. Comparison of effects of LAT8881 and LAT7771, via two administration routes (orally (PO) or intramuscularly (IM)) on PWT in Chung model rats, on the ipsilateral side of the injury. Both LAT8881 and LAT7771 administered via oral or intramuscular routes significantly increased the PWT in Chung model rats. B. Comparison of effects of LAT8881 and LAT7771, via two administration routes (orally (PO) or intramuscularly (IM)) on PWT in Chung model rats on the contralateral side of the injury.

### DETAILED DESCRIPTION OF THE INVENTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an peptide” means one peptide or more than one peptide.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

Methods of treatment

The present invention is predicated, at least in part, on the inventors' surprising finding that prolactin-derived peptides of formula (I) (SEQ ID NO:1) have advantageous analgesic properties, in that they are capable of alleviating neuropathic pain. Thus, in an aspect disclosed herein, there is provided a method of treating and preventing neuropathic pain in a subject, the method comprising administering to a subject a therapeutically effective amount of prolactin, or a functional variant thereof, wherein the functional variant comprises a peptide of formula (I), or a pharmaceutically acceptable salt thereof:

(SEQ ID NO: 1)
R¹-C-R-I-X₁-X₂-X₃-X₄-N-C-R² (I)

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent.

In a preferred embodiment, the peptide of formula (I) is CRIIHNNNC (SEQ ID NO:2). SEQ ID NO:2 (referred to interchangeably herein as LAT7771) is the C-terminal fragment of human prolactin (PRL) spanning amino acid residues 219-227 of human prolactin precursor (hPRL) (see, e.g., NCBI Reference sequence NP 000939.1 and NP 001157030).

The term “functional variant” is used herein to denote a peptide that differs structurally from the native prolactin peptide (e.g., human prolactin of SEQ ID NO:30, as herein described) but retains at least some or all of the biological activity of the native prolactin in the treatment or prevention of neuropathic pain. The term “functional variant” includes insertions, deletions and/or substitutions, either conservative or non-conservative, where such changes do not substantially alter the ability of the variant to treat or prevent neuropathic pain when administered to a subject in need thereof, as described herein. Suitable methods for determining whether the variant is a functional variant of prolactin will be familiar to persons skilled in the art, illustrative examples of which are described elsewhere herein (e.g, the ability of the functional variant to modify neuronal signaling ex vivo). Functional variants also extend to non-human isoforms of prolactin, including functional fragments thereof. Non-human isoforms of prolactin will be known to persons skilled in the art, illustrative examples of which are described in Hashimoto et al. (2010, Exp Anim; 59 (5) 643-6) and Miller et al. (1981, DNA; 1(1) 37-50), the contents of which are incorporated herein by reference in their entirety.

In an embodiment disclosed herein, the functional variant of prolactin is a functional fragment of native prolactin (SEQ ID NO:30). A functional fragment of native prolactin can be any suitable length, as long as the fragment retains at least some analgesic properties. In an embodiment, the functional fragment is up to 50 amino acid residues in length, preferably up to 45 amino acid residues in length, preferably up to 40 amino acid residues in length, preferably up to 35 amino acid residues in length, preferably up to 30 amino acid residues in length, preferably up to 25 amino acid residues in length, preferably up to 20 amino acid residues in length, preferably up to 15 amino acid residues in length, or more preferably up to 10 amino acid residues in length.

The functional variant may include a molecule that has an amino acid sequence that differs from the amino acid sequence of native prolactin by one or more (e.g., 1, 2, 3, 4, 5, 6, 7, 8, 9 or more) amino acid substitutions, wherein said difference does not, or does not completely abrogate the ability of the variant to alleviate neuropathic pain when administered to a subject in need thereof. In some embodiments, the functional variant comprises amino acid substitutions that enhance the ability of the variant to alleviate neuropathic pain when administered to a subject in need thereof. In an embodiment, the functional variant has an amino acid sequence that differs from the amino acid sequence of native prolactin by one or more conservative amino acid substitutions. As used herein, the term “conservative amino acid substitution” refers to changing amino acid identity at a given position to replace it with an amino acid of approximately equivalent size, charge and/or polarity. Examples of natural conservative substitutions of amino acids include the following 8 substitution groups (designated by the conventional one-letter code): (1) M, I, L, V; (2) F, Y, W; (3) K, R, (4) A, G; (5) S, T; (6) Q, N; (7) E, D; and (8) C, S.

In an embodiment, the functional variant has at least 85% sequence identity to an amino acid sequence of native prolactin. Reference to “at least 85%” includes 85%, 86%, 87%, 88%, 89%, 90%, 91%, 92%, 93%, 94%, 95%, 96%, 97%, 98%, 99% or 100% sequence identity or similarity, for example, after optimal alignment or best fit analysis. Thus, in an embodiment, the sequence has at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity or sequence homology with the sequences identified herein, for example, after optimal alignment or best fit analysis.

The terms “identity”, “similarity”, “sequence identity”, “sequence similarity”, “homology”, “sequence homology” and the like, as used herein, mean that at any particular amino acid residue position in an aligned sequence, the amino acid residue is identical between the aligned sequences. The term “similarity” or “sequence similarity” as used herein, indicates that, at any particular position in the aligned sequences, the amino acid residue is of a similar type between the sequences. For example, leucine may be substituted for an isoleucine or valine residue. As noted elsewhere herein, this may be referred to as conservative substitution. In an embodiment, an amino acid sequence may be modified by way of conservative substitution of any of the amino acid residues contained therein, such that the modification has no effect on the ability of the functional variant to alleviate pain when administered to a subject in need thereof when compared to the unmodified (native) prolactin peptide/protein.

In some embodiments, sequence identity with respect to a peptide sequence relates to the percentage of amino acid residues in the candidate sequence which are identical with the residues of the corresponding peptide sequence after aligning the sequences and introducing gaps, if necessary, to achieve the maximum percentage homology, and not considering any conservative substitutions as part of the sequence identity. Neither N- or C-terminal extensions, nor insertions shall be construed as reducing sequence identity or homology. Methods and computer programs for performing an alignment of two or more amino acid sequences and determining their sequence identity or homology are well known to persons skilled in the art. For example, the percentage of identity or similarity of two amino acid sequences can be readily calculated using algorithms, for example, BLAST, FASTA, or the Smith-Waterman algorithm.

Techniques for determining an amino acid sequence “similarity” are well known to persons skilled in the art. In general, “similarity” means an exact amino acid to amino acid comparison of two or more peptide sequences or at the appropriate place, where amino acids are identical or possess similar chemical and/or physical properties such as charge or hydrophobicity. A so-termed “percent similarity” then can be determined between the compared peptide sequences. In general, “identity” refers to an exact amino acid to amino acid correspondence of two peptide sequences.

Two or more peptide sequences can also be compared by determining their “percent identity”. The percent identity of two sequences may be described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff (Atlas of Protein Sequences and Structure, M. O. Dayhoff ed., 5 suppl. 3:353-358, National Biomedical Research Foundation, Washington, D.C., USA), and normalized by Gribskov (Nucl. Acids Res. 14(6):6745-6763, 1986). Suitable programs for calculating the percent identity or similarity between sequences are generally known in the art.

Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., (1997, Nucl. Acids Res.25:3389). A detailed discussion of sequence analysis can be found in Unit 19.3 of Ausubel et al. (“Current Protocols in Molecular Biology”, John Wiley & Sons Inc, 1994-1998, Chapter 15).

In an embodiment, a functional variant includes amino acid substitutions and/or other modifications relative to native prolactin in order to increase the stability of the variant or to increase the solubility of the variant.

The functional variant may be a naturally-occurring peptide or it may be synthetically produced by chemical synthesis using methods known to persons skilled in the art.

In an embodiment, the functional variant comprises an amino acid sequence having at least 80% sequence identity to SEQ ID NO:30. In another embodiment, the functional variant is a functional fragment of native prolactin. In an embodiment disclosed herein, R¹ is absent. In another embodiment, R² is absent. In yet another embodiment, R¹ and R² are absent.

The present inventors have also surprisingly found that the analgesic property of the peptides described herein is retained when the peptide of formula (I) is modified by a C-terminal extension. For example, the inventors have surprisingly found that SEQ ID NO:3 (CRIIHNNNCG; also referred to herein as 7772) has a therapeutically effective analgesic effect on neuropathic pain. Thus, in an embodiment, the functional variant comprises a C-terminal amino acid residue. In an embodiment, the C-terminal amino acid residue is glycine (G).

In an embodiment disclosed herein, the functional variant is from 9 to 165 amino acid residues in length, preferably at least 9 amino acid residues in length, preferably at least 10 amino acid residues in length, preferably at least 11 amino acid residues in length, preferably at least 12 amino acid residues in length, preferably at least 13 amino acid residues in length, preferably at least 14 amino acid residues in length, preferably at least 15 amino acid residues in length, or more preferably at least 16 amino acid residues in length. The prolactin, or functional variant thereof, comprises a disulphide bond between the two cysteine (C) residues at positions 2 and 10 of formula (I), thereby forming a cyclic peptide between the two cysteine residues.

In an embodiment, the functional variant is selected from the group consisting of CRIIHNNNC (SEQ ID NO:2), CRIIHNNNCG (SEQ ID NO:3), CRIVYDSNC (SEQ ID NO:4) and CRIVYDSNCG (SEQ ID NO:5).

In another embodiment, the functional variant is selected from the group consisting of YLKLLKCRIIHNNNC (SEQ ID NO:6), LKLLKCRIIHNNNC (SEQ ID NO:7), KLLKCRIIHNNNC (SEQ ID NO:8), LLKCRIIHNNNC (SEQ ID NO:9), LKCRIIHNNNC (SEQ ID NO:10), KCRIIHNNNC (SEQ ID NO:11), YLKLLKCRIIHNNNCG (SEQ ID NO:12), LKLLKCRIIHNNNCG (SEQ ID NO:13), KLLKCRIIHNNNCG (SEQ ID NO:14), LLKCRIIHNNNCG (SEQ ID NO:15), LKCRIIHNNNCG (SEQ ID NO:16) and KCRIIHNNNCG (SEQ ID NO:17).

In an embodiment, the functional variant is CRIIHNNNC (SEQ ID NO:2). In another embodiment, the functional variant is CRIIHNNNCG (SEQ ID NO:3).

The present inventors have also surprisingly found that non-human variants of prolactin-derived peptides comprising the peptide of formula (I) have similar analgesic properties to their human counterparts. Suitable non-human variants will be familiar to persons skilled in the art, illustrative examples of which include SEQ ID NOs:4 and 5.

In an aspect disclosed herein, there is provided a method of treating and preventing neuropathic pain in a subject, the method comprising administering to a subject a therapeutically effective amount of a peptide of SEQ ID NO:4 (CRIVYDSNC) or SEQ ID NO:5 (CRIVYDSNCG). SEQ ID NO:4 is an illustrative example of a C-terminal fragment of canine prolactin, spanning amino acid residues 221-229 of canis lupus familiaris prolactin precursor (see, e.g. GenBank Accession ADK11290).

In an embodiment, the functional variant is selected from the group consisting of CRIVYDSNCG (SEQ ID NO:5), YLKLLKCRIVYDSNC (SEQ ID NO:18), LKLLKCRIVYDSNC (SEQ ID NO:19), KLLKCRIVYDSNC (SEQ ID NO:20), LLKCRIVYDSNC (SEQ ID NO:21), LKCRIVYDSNC (SEQ ID NO:22), KCRIVYDSNC (SEQ ID NO:23), YLKLLKCRIVYDSNCG (SEQ ID NO:24), LKLLKCRIVYDSNCG (SEQ ID NO:25), KLLKCRIVYDSNCG (SEQ ID NO:26), LLKCRIVYDSNCG (SEQ ID NO:27), LKCRIVYDSNCG (SEQ ID NO:28), and KCRIVYDSNCG (SEQ ID NO:29)

In an embodiment, the functional variant is CRIVYDSNC (SEQ ID NO:4). In another preferred embodiment, the functional variant is CRIVYDSNCG (SEQ ID NO:5).

The peptides of formula (I) may be made of naturally occurring amino acid residues, proteogenic or non-proteogenic. These amino acids have L-stereochemistry. Naturally occurring amino acids are set out in the table below.

(1)

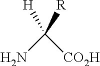

(2)

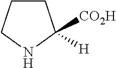

| | Three-letter | One-letter | Structure of side chain (R) |
|---|---|---|---|
| Amino Acid | Abbreviation | symbol | in (1) above |
| Alanine | Ala | A | —CH₃ |
| Arginine | Arg | R | —(CH₂)₃NHC(=N)NH₂ |
| Asparagine | Asn | N | —CH₂CONH₂ |
| Aspartic acid | Asp | D | —CH₂CO₂H |
| Cysteine | Cys | C | —CH₂SH |
| Glutamine | Gln | Q | —(CH₂)₂CONH₂ |
| Glutamic acid | Glu | E | —(CH₂)₂CO₂H |
| Glycine | Gly | G | —H |
| Histidine | His | H | —CH₂(4-imidazolyl) |
| Isoleucine | Ile | I | —CH(CH₃)CH₂CH₃ |
| Leucine | Leu | L | —CH₂CH(CH₃)₂ |
| Lysine | Lys | K | —(CH₂)₄NH₂ |
| Methionine | Met | M | —(CH₂)₂SCH₃ |
| Phenylalanine | Phe | F | —CH₂Ph |
| Ornithine | Orn | O | —(CH₂)₃NH₂ |
| Proline | Pro | P | see formula (2) above for |
| | | | structure of amino acid |
| Serine | Ser | S | —CH₂OH |
| Threonine | Thr | T | —CH(CH₃)OH |
| Tryptophan | Trp | W | —CH₂(3-indolyl) |
| Tyrosine | Tyr | Y | —CH₂(4-hydroxyphenyl) |
| Valine | Val | V | —CH(CH₃)₂ |

As used herein, the term “alkyl” refers to a straight chain or branched saturated hydrocarbon group having 1 to 10 carbon atoms. Where appropriate, the alkyl group may have a specified number of carbon atoms, for example, C1-6alkyl which includes alkyl groups having 1, 2, 3, 4, 5 or 6 carbon atoms in a linear or branched arrangement. Examples of suitable alkyl groups include, but are not limited to, methyl, ethyl, n-propyl, i-propyl, n-butyl, i-butyl, t-butyl, n-pentyl, 2-methylbutyl, 3-methylbutyl, 4-methylbutyl, n-hexyl, 2-methylpentyl, 3-methylpentyl, 4-methylpentyl, 5-methylpentyl, 2-ethylbutyl, 3-ethylbutyl, heptyl, octyl, nonyl and decyl.

As used herein, the term “alkenyl” refers to a straight-chain or branched hydrocarbon group having one or more double bonds between carbon atoms and having 2 to 10 carbon atoms. Where appropriate, the alkenyl group may have a specified number of carbon atoms. For example, C2-C6 as in “C2-C6alkenyl” includes groups having 2, 3, 4, 5 or 6 carbon atoms in a linear or branched arrangement. Examples of suitable alkenyl groups include, but are not limited to, ethenyl, propenyl, isopropenyl, butenyl, butadienyl, pentenyl, pentadienyl, hexenyl, hexadienyl, heptenyl, octenyl, nonenyl and decenyl.

As used herein, the term “alkynyl” refers to a straight-chain or branched hydrocarbon group having one or more triple bonds and having 2 to 10 carbon atoms. Where appropriate, the alkynyl group may have a specified number of carbon atoms. For example, C2-C6 as in “C2-C6alkynyl” includes groups having 2, 3, 4, 5 or 6 carbon atoms in a linear or branched arrangement. Examples of suitable alkynyl groups include, but are not limited to ethynyl, propynyl, butynyl, pentynyl and hexynyl.

As used herein, the term “cycloalkyl” refers to a saturated and unsaturated (but not aromatic) cyclic hydrocarbon. The cycloalkyl ring may include a specified number of carbon atoms. For example, a 3 to 8 membered cycloalkyl group includes 3, 4, 5, 6, 7 or 8 carbon atoms. Examples of suitable cycloalkyl groups include, but are not limited to, cyclopropyl, cyclobutyl, cyclopentyl, cyclopentenyl, cyclohexyl, cyclohexenyl, cyclohexadienyl, cycloheptyl and cyclooctyl.

As used herein, the term “aryl” is intended to mean any stable, monocyclic, bicyclic or tricyclic carbon ring system of up to 7 atoms in each ring, wherein at least one ring is aromatic. Examples of such aryl groups include, but are not limited to, phenyl, naphthyl, tetrahydronaphthyl, indanyl, fluorenyl, phenanthrenyl, biphenyl and binaphthyl.

In an embodiment, a disulphide bond is formed between the two cysteine residues (C) of formula (I).

In an embodiment, the prolactin or functional variant thereof is formed as a pharmaceutically acceptable salt. It is to be understood that non-pharmaceutically acceptable salts are also envisaged, since these may be useful as intermediates in the preparation of pharmaceutically acceptable salts or may be useful during storage or transport. Suitable pharmaceutically acceptable salts will be familiar to persons skilled in the art, illustrative examples of which include salts of pharmaceutically acceptable inorganic acids, such as hydrochloric, sulphuric, phosphoric, nitric, carbonic, boric, sulfamic, and hydrobromic acids, or salts of pharmaceutically acceptable organic acids, such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicylic sulphanilic, aspartic, glutamic, edetic, stearic, palmitic, oleic, lauric, pantothenic, tannic, ascorbic and valeric acids. Illustrative examples of suitable base salts include those formed with pharmaceutically acceptable cations, such as sodium, potassium, lithium, calcium, magnesium, ammonium and alkylammonium. Basic nitrogen-containing groups may be quaternized with such agents as lower alkyl halide, such as methyl, ethyl, propyl, and butyl chlorides, bromides and iodides; dialkyl sulfates like dimethyl and diethyl sulfate; and others.

Also disclosed herein are prodrugs comprising prolactin, or a functional variant thereof, as herein described. As used herein, a “prodrug” typically refers to a compound that can be metabolized in vivo to provide the active peptide of formula (I), or pharmaceutically acceptable salts thereof. In some embodiments, the prodrug itself also shares the same, or substantially the same, analgesic activity as prolactin, or a functional variant, as described elsewhere herein.

In some embodiments, prolactin, or a functional variant thereof, may further comprise a C-terminal capping group. The term “C-terminal capping group”, as used herein, refers to a group that blocks the reactivity of the C-terminal carboxylic acid. Suitable C-terminal capping groups form amide groups or esters with the C-terminal carboxylic acid, for example, the C-terminal capping group forms a —C(O)NHR^{a} or —C(O)OR^{b} where the C(O) is from the C-terminal carboxylic acid group and R^{a} is hydrogen, alkyl, alkenyl, alkynyl, cycloalkyl or aryl and R^{b} is alkyl, alkenyl, alkynyl, cycloalkyl or aryl. In particular embodiments, the C-terminal capping group is —NH₂, forming —C(O)NH₂. In some embodiments, the peptides of formula (I), or pharmaceutically acceptable salts thereof, comprise a C-terminal polyethylene glycol (PEG). In an embodiment, the PEG has a molecular weight in the range of 220 to 5500 Da, preferably 220 to 2500 Da, more preferably 570 to 1100 Da.

In some embodiments, the prolactin, or functional variant thereof, may further comprise an N-terminal capping group. The term “N-terminal capping group”, as used herein, refers to a group that blocks the reactivity of the N-terminal amino group. Suitable N-terminal capping groups are acyl groups that form amide groups with the N-terminal amino group, for example, the N-terminal capping group forms a —NHC(O)R^{a} where the NH is from the N-terminal amino group and R^{a} is alkyl, alkenyl, alkynyl, cycloalkyl or aryl. In particular embodiments, the N-terminal capping group is —C(O)CH₃ (acyl), forming —NHC(O)CH₃.

In some embodiments, the prolactin, or functional variant thereof, may comprise a C-terminal capping group and an N-terminal capping group, as herein described.

Prolactin, or functional variants thereof as herein described, can be made by any method known to persons skilled in the art. Illustrative examples of suitable methods include solution or solid phase synthesis using Fmoc or Boc protected amino acid residues, recombinant techniques using microbial culture, genetically engineered microbes, plants and recombinant DNA technology (see, e.g., Sambrook and Russell, Molecular Cloning: A Laboratory Manual (3^{rd} Edition), 2001, CSHL Press).

As described elsewhere herein, present inventors have surprisingly found, for the first time, that prolactin-derived peptides comprising the peptide of formula (I) (SEQ ID NO:1) have analgesic properties, insofar as they are capable of alleviating neuropathic pain. Prolactin, or functional variants thereof, as herein described, can therefore suitably be used to treat, prevent, alleviate or otherwise delay the onset of neuropathic pain in a subject, including one or more symptoms of neuropathic pain. The present inventors have also surprisingly found that non-human prolactin-derived peptides comprising the peptide of formula (I) have similar analgesic properties to their human counterparts.

Prolactin, or functional variants thereof, can therefore suitably be used to treat, prevent, alleviate or otherwise delay the onset of neuropathic pain in a subject, including one or more symptoms of neuropathic pain.

The terms “treating”, “treatment” and the like, are used interchangeably herein to mean relieving, reducing, alleviating, ameliorating or otherwise inhibiting neuropathic pain, including one or more symptoms of neuropathic pain, such as allodynia or hyperalgesia. The terms “prevent”, “preventing”, “prophylaxis”, “prophylactic”, “preventative” and the like are used interchangeably herein to mean preventing or delaying the onset of neuropathic pain, or the risk of developing neuropathic pain.

The terms “treating”, “treatment” and the like also include relieving, reducing, alleviating, ameliorating or otherwise inhibiting the effects of the neuropathic pain for at least a period of time. It is also to be understood that terms “treating”, “treatment” and the like do not imply that the neuropathic pain, or a symptom thereof, is permanently relieved, reduced, alleviated, ameliorated or otherwise inhibited and therefore also encompasses the temporary relief, reduction, alleviation, amelioration or otherwise inhibition of neuropathic pain, or a symptom thereof.

Without being bound by theory, or by a particular mode of application, neuropathic pain is typically characterised as pain which results from damage by injury or disease to nerve tissue or neurones per se or of dysfunction within nerve tissue. The neuropathic pain may be peripheral, central or a combination thereof; in other words, the term “neuropathic pain” typically refers to any pain syndrome initiated or caused by a primary lesion or dysfunction in the peripheral or central nervous system. Neuropathic pain is also distinguishable in that it typically does not respond effectively to treatment by common pain medication such as opioids. By contrast, nociceptive pain is characterised as pain which results from stimulation of nociceptors by noxious or potentially harmful stimuli that may cause damage or injury to tissue. Nociceptive pain is typically responsive to common pain medication, such as opioids.

The term “analgesia” is used herein to describe states of reduced pain perception, including absence from pain sensations, as well as states of reduced or absent sensitivity to noxious stimuli. Such states of reduced or absent pain perception are typically induced by the administration of a pain-controlling agent or agents and occur without loss of consciousness, as is commonly understood in the art. Suitable methods for determining whether a compound is capable of providing an analgesic effect will be familiar to persons skilled in the art, illustrative examples of which include the use of animal models of neuropathic pain, such as chronic constriction injury, spinal nerve ligation and partial sciatic nerve ligation (see Bennett et al. (2003); Curr. Protoc. Neurosci., Chapter 9, Unit 9.14) and animal models of nociceptive pain, such as formalin-, carrageenan- or complete Freund's adjuvant (CFA)-induced inflammatory pain. Other suitable models of neuropathic pain are discussed in Gregory et al. (2013, J. Pain.; 14(11); “:An overview of animal models of pain: disease models and outcome measures”).

As persons skilled in the art will know, there are many possible causes of neuropathy and neuropathic pain. It is therefore to be understood that contemplated herein is the treatment or prevention of neuropathic pain regardless of cause. In some embodiments, pain is a result of injury or trauma to tissue, disease or condition affecting the nerves (e.g., primary neuropathy) and/or pain that is caused by systemic disease (secondary neuropathy), illustrative examples of which include diabetic neuropathy; Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HIV sensory neuropathies; hereditary motor and sensory neuropathies (HMSN); hereditary sensory neuropathies (HSNs); hereditary sensory and autonomic neuropathies; hereditary neuropathies with ulcero-mutilation; nitrofurantoin neuropathy; tomaculous neuropathy; neuropathy caused by nutritional deficiency, neuropathy caused by kidney failure and complex regional pain syndrome. Other illustrative examples of conditions that may cause neuropathic pain include repetitive activities such as typing or working on an assembly line, medications known to cause peripheral neuropathy such as several antiretroviral drugs ddC (zalcitabine) and ddI (didanosine), antibiotics (metronidazole, an antibiotic used for Crohn's disease, isoniazid used for tuberculosis), gold compounds (used for rheumatoid arthritis), some chemotherapy drugs (such as vincristine and others) and many others. Chemical compounds are also known to cause peripheral neuropathy including alcohol, lead, arsenic, mercury and organophosphate pesticides. Some peripheral neuropathies are associated with infectious processes (such as Guillain-Barré syndrome). Other illustrative examples of neuropathic pain include thermal or mechanical hyperalgesia, thermal or mechanical allodynia, diabetic pain, neuropathic pain affecting the oral cavity (e.g., trigeminal neuropathic pain, atypical odontalgia (phantom tooth pain), burning mouth syndrome), fibromyalgia and entrapment pain.

In an embodiment disclosed herein, the neuropathic pain is associated with a condition selected from the group consisting of diabetic neuropathy; Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HIV sensory neuropathy; hereditary motor and sensory neuropathy (HMSN); hereditary sensory neuropathy (HSN); hereditary sensory and autonomic neuropathy; hereditary neuropathy with ulcero-mutilation; nitrofurantoin neuropathy; tomaculous neuropathy; neuropathy caused by nutritional deficiency, neuropathy caused by kidney failure, trigeminal neuropathic pain, atypical odontalgia (phantom tooth pain), burning mouth syndrome, complex regional pain syndrome, repetitive strain injury, drug-induced peripheral neuropathy. peripheral neuropathy associated with infection, allodynia, hyperesthesia and hyperalgesia. In another embodiment, the neuropathic pain is burning pain or shooting pain.

In some embodiments, the neuropathic pain may be accompanied by numbness, weakness and loss of reflexes. The neuropathic pain may be severe and disabling. By “hyperalgesia” is meant an increased response to a stimulus that is normally painful. A hyperalgesia condition is one that is associated with pain caused by a stimulus that is not normally painful. The term “hyperesthesia” refers to an excessive physical sensitivity, especially of the skin. The term “allodynia” as used herein refers to the pain that results from a non-noxious stimulus; that is, pain due to a stimulus that does not normally provoke pain. Illustrative examples of allodynia include thermal allodynia (pain due to a cold or hot stimulus), tactile allodynia (pain due to light pressure or touch), mechanical allodynia (pain due to heavy pressure or pinprick) and the like.

Neuropathic pain may be acute or chronic and, in this context, it is to be understood that the time course of a pain may vary, based on its underlying cause. For instance, with trauma, the onset of symptoms of neuropathic pain may be acute, or sudden; however, the most severe symptoms may develop over time and persist for years. A chronic time course over weeks to months usually indicates a toxic or metabolic pain syndrome. A chronic, slowly progressive pain syndrome, such as occurs with painful diabetic neuropathy or with most hereditary neuropathies or with a condition termed chronic inflammatory demyelinating polyradiculoneuropathy (CIDP), may have a time course over many years. Neuropathic conditions with symptoms that relapse and remit include Guillain-Barré syndrome.

In some embodiments, neuropathic pain results from a condition characterised by neuronal hypersensitivity, such as fibromyalgia or irritable bowel syndrome.

In other embodiments, neuropathic pain results from a disorder associated with aberrant nerve regeneration resulting in neuronal hypersensitivity. Such disorders include breast pain, interstitial cystitis, vulvodynia and cancer chemotherapy-induced neuropathy.

In some embodiments, the neuropathic pain is related to surgery, pre-operative pain and post-operative pain, particularly post-operative pain.

The term “subject”, as used herein, refers to a mammalian subject for whom treatment or prophylaxis of neuropathic pain is desired. Illustrative examples of suitable subjects include primates, especially humans, companion animals such as cats and dogs and the like, working animals such as horses, donkeys and the like, livestock animals such as sheep, cows, goats, pigs and the like, laboratory test animals such as rabbits, mice, rats, guinea pigs, hamsters and the like and captive wild animals such as those in zoos and wildlife parks, deer, dingoes and the like. In an embodiment, the subject is a human. In another embodiment, the subject is selected from the group consisting of a canine, a feline and an equine.

It is to be understood that a reference to a subject herein does not imply that the subject has neuropathic pain, or a symptom thereof, but also includes a subject that is at risk of developing neuropathic pain, or a symptom thereof. In an embodiment, the subject has (i.e., is experiencing) neuropathic pain or a symptom thereof. In another embodiment, the subject is not experiencing neuropathic pain or a symptom thereof at the time of treatment, but is at risk of developing neuropathic pain or a symptom thereof. In an illustrative example, the subject has a disease or condition that puts the subject at risk of developing neuropathic pain, for example, poorly managed diabetes, which may lead to a diabetic neuropathy. In another embodiment, the subject has had a disease or condition that has potential to result in neuropathic pain, such as herpes zoster (shingles), which may lead to post-herpetic neuralgia.

In some embodiments, the methods disclosed herein comprise administering prolactin, or a functional variant thereof having at least 80% sequence identity to SEQ ID NO:30 wherein the functional variant comprises a peptide of formula (I), or a pharmaceutically acceptable salt thereof, to a non-human subject. In a preferred embodiment, the non-human subject is selected from the group consisting of a canine, a feline or an equine. In other embodiments, the methods disclosed herein comprise administering prolactin, or a functional variant thereof having at least 80% sequence identity to SEQ ID NO:30 wherein the functional variant comprises a peptide of formula (I), or a pharmaceutically acceptable salt thereof, to a human subject. In other embodiments, prolactin, or a functional variant thereof having at least 80% sequence identity to SEQ ID NO:30 wherein the functional variant comprises a peptide of formula (I), or a pharmaceutically acceptable salt thereof, is administered to a non-human subject, such as a canine, a feline or an equine.

Prolactin, or a functional variant thereof having at least 80% sequence identity to SEQ ID NO:30 wherein the functional variant comprises a peptide of formula (I), or pharmaceutically acceptable salts thereof, are to be administered in a therapeutically effective amount. The phrase “therapeutically effective amount” typically means an amount necessary to attain the desired response, or to delay the onset or inhibit progression or halt altogether, the onset or progression of neuropathic pain being treated. It would be understood by persons skilled in the art that the therapeutically effective amount of peptide will vary depending upon several factors, illustrative examples of which include the health and physical condition of the subject to be treated, the taxonomic group of subject to be treated, the severity of the neuropathic pain to be treated, the formulation of the composition comprising a peptide of formula (I), or a pharmaceutically acceptable salt thereof, the route of administration, and combinations of any of the foregoing.

The therapeutically effective amount will typically fall within a relatively broad range that can be determined through routine trials by persons skilled in the art. Illustrative examples of a suitable therapeutically effective amount of the prolactin, or functional variant thereof, for administration to a human subject include from about 0.001 mg per kg of body weight to about 1 g per kg of body weight, preferably from about 0.001 mg per kg of body weight to about 50g per kg of body weight, more preferably from about 0.01 mg per kg of body weight to about 1.0 mg per kg of body weight. In an embodiment disclosed herein, the therapeutically effective amount of the peptide of formula (I), or pharmaceutically acceptable salts thereof, is from about 0.001 mg per kg of body weight to about 1 g per kg of body weight per dose (e.g., 0.001 mg/kg, 0.005 mg/kg, 0.01 mg/kg, 0.05 mg/kg, 0.1 mg/kg, 0.15 mg/kg, 0.2 mg/kg, 0.25 mg/kg, 0.3 mg/kg, 0.35 mg/kg, 0.4 mg/kg, 0.45 mg/kg, 0.5 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, lmg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 10.5 mg/kg, llmg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 16.5 mg/kg, 17 mg/kg, 17.5 mg/kg, 18 mg/kg, 18.5 mg/kg, 19 mg/kg, 19.5 mg/kg, 20 mg/kg, 20.5 mg/kg, 21 mg/kg, 21.5 mg/kg, 22 mg/kg, 22.5 mg/kg, 23 mg/kg, 23.5 mg/kg, 24 mg/kg, 24.5 mg/kg, 25 mg/kg, 25.5 mg/kg, 26 mg/kg, 26.5 mg/kg, 27 mg/kg, 27.5 mg/kg, 28 mg/kg, 28.5 mg/kg, 29 mg/kg, 29.5 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg of body weight, etc). In an embodiment, the therapeutically effective amount of the peptides of formula (I), or the pharmaceutically acceptable salts thereof, is from about 0.001 mg to about 50 mg per kg of body weight. In an embodiment, the therapeutically effective amount of the peptides of formula (I), or the pharmaceutically acceptable salts thereof, is from about 0.01 mg to about 1.0 mg per kg of body weight. Dosage regimes may be adjusted to provide the optimum therapeutic response. For example, several divided doses may be administered daily, weekly, monthly or other suitable time intervals, or the dose may be proportionally reduced as indicated by the exigencies of the situation.

In an embodiment disclosed herein, the peptide of formula (I), or the pharmaceutically acceptable salt thereof, is administered to the subject at a therapeutically effective amount that alleviates neuropathic pain in the subject with minimal or no analgesic effect on nociceptive pain. The present inventors have also surprisingly found that non-human variants of prolactin, or functional variants thereof have similar analgesic properties to their human counterparts.

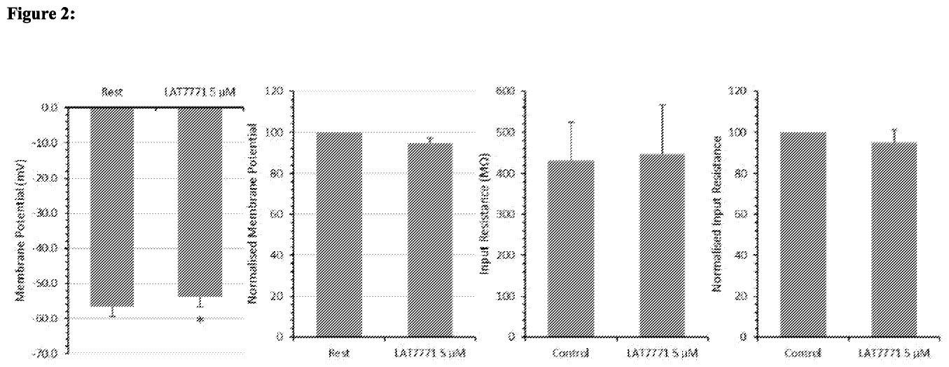

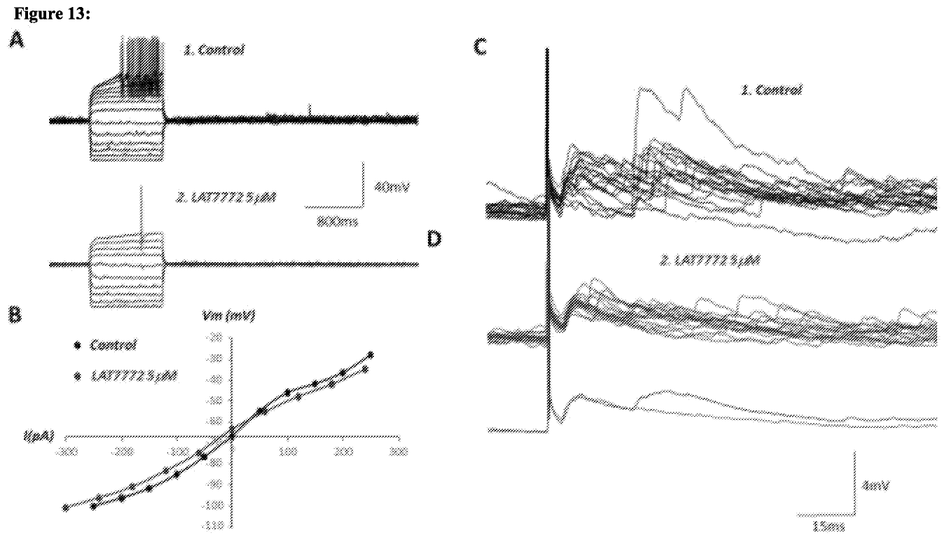

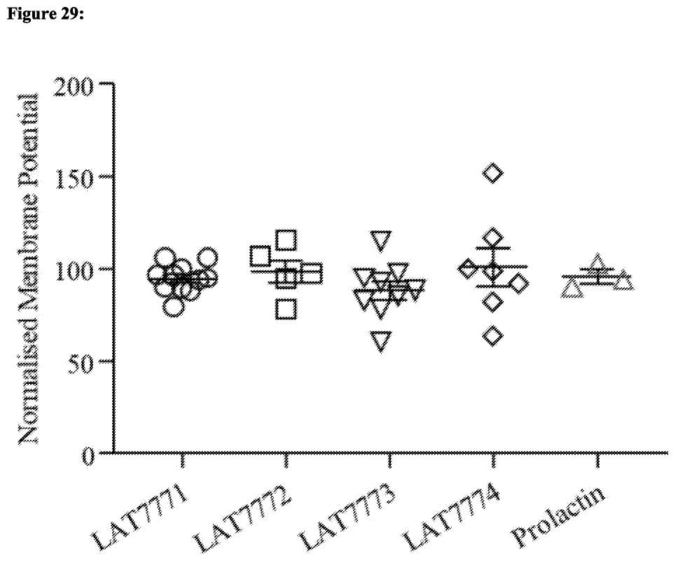

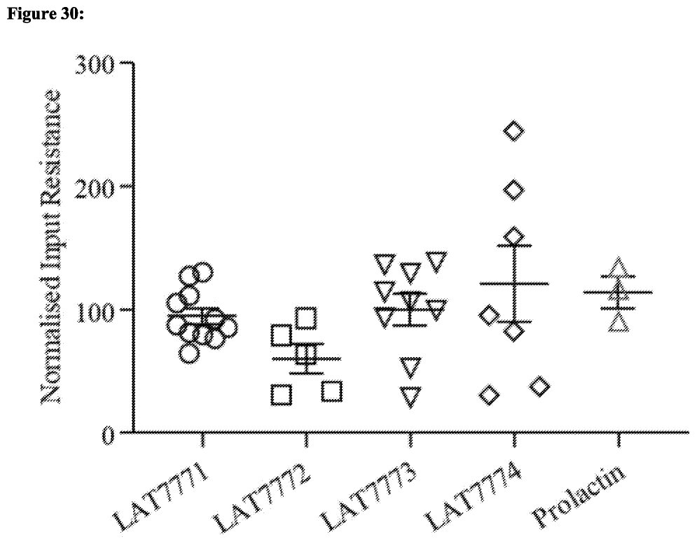

The analgesic properties are evident from the data derived from the nerve constriction model of neuropathic pain (see FIGS. 2-30). As can be seen from that data, C-terminal fragments of prolactin (SEQ ID NO:2-5) showed comparable activity to the parent prolactin protein in terms of both magnitude and duration of neuropathic analgesia (FIG. 29-30). Thus, in another aspect disclosed herein, there is provided a method of treating neuropathic pain in a subject, the method comprising administering to a subject a therapeutically effective amount of a peptide, or a pharmaceutically acceptable salt thereof, wherein the peptide comprises, consists, or consists essentially, of amino acid sequence CRIIHNNNC (SEQ ID NO:2), CRIIHNNNCG (SEQ ID NO:3), CRIVYDSNC (SEQ ID NO:4) and CRIVYDSNCG (SEQ ID NO:5).

### Routes of Administration

The peptides disclosed herein may be administered to the subject by any suitable route that allows for delivery of the peptides to the subject at a therapeutically effective amount, as herein described. Suitable routes of administration will be known to persons skilled in the art, illustrative examples of which include enteral routes of administration (e.g., oral and rectal), parenteral routes of administration, typically by injection or microinjection (e.g., intramuscular, subcutaneous, intravenous, epidural, intra-articular, intraperitoneal, intracisternal or intrathecal) and topical (transdermal or transmucosal) routes of administration (e.g., buccal, sublingual, vaginal, intranasal or by inhalation). The peptides disclosed herein may also suitably be administered to the subject as a controlled release dosage form to provide a controlled release of the active agent(s) over an extended period of time. The term “controlled release” typically means the release of the active agent(s) to provide a constant, or substantially constant, concentration of the active agent in the subject over a period of time (e.g., about eight hours up to about 12 hours, up to about 14 hours, up to about 16 hours, up to about 18 hours, up to about 20 hours, up to a day, up to a week, up to a month, or more than a month). Controlled release of the active agent(s) can begin within a few minutes after administration or after expiration of a delay period (lag time) after administration, as may be required. Suitable controlled release dosage forms will be known to persons skilled in the art, illustrative examples of which are described in Anal, A. K. (2010; Controlled-Release Dosage Forms. Pharmaceutical Sciences Encyclopedia. 11:1-46).

Without being bound by theory or by a particular mode of application, it may be desirable to elect a route of administration on the basis of whether the neuropathic pain is localized or generalised. For example, where the neuropathic pain is localized, it may be desirable to administer the peptides to the affected area or to an area immediately adjacent thereto. For instance, where the neuropathic pain is in a joint (e.g., neck, knee, elbow, shoulder, hip, etc.), the peptides can be administered to the subject intra-articularly into the affected joint. Alternatively, or in addition, the peptides can be administered at, or substantially adjacent to, the affected joint. As another illustrative example, where the neuropathic pain is in the oral cavity (e.g., trigeminal neuropathic pain, atypical odontalgia (phantom tooth pain) or burning mouth syndrome), the peptides can be formulated for administration via the oral mucosa (e.g., by buccal and/or sublingual administration). Conversely, where the neuropathic pain is generalised or disseminated across multiple anatomical sites of a subject, the peptides may be administered topically, enterally and/or parenterally at any site with a view to distributing the active peptides across the multiple anatomical sites affected by neuropathic pain. In an embodiment disclosed herein, the peptides disclosed herein are administered to the subject enterally. In an embodiment disclosed herein, the peptides disclosed herein are administered to the subject orally. In an embodiment disclosed herein, the peptides disclosed herein are administered to the subject parenterally. In another embodiment disclosed herein, the peptides disclosed herein are administered to the subject topically. As described elsewhere herein, “topical” administration typically means application of the active agents to a surface of the body, such as the skin or mucous membranes, suitably in the form of a cream, lotion, foam, gel, ointment, nasal drop, eye drop, ear drop, transdermal patch, transdermal film (e.g., sublingual film) and the like. Topical administration also encompasses administration via the mucosal membrane of the respiratory tract by inhalation or insufflation. In an embodiment disclosed herein, the topical administration is selected from the group consisting of transdermal and transmucosal administration. In an embodiment, the peptides disclosed herein are administered to the subject transdermally.

In an embodiment, the methods comprise orally administering the peptides disclosed herein to a human. In another embodiment, the methods comprise orally administering the peptides disclosed herein to a non-human subject. In yet another embodiment, the methods comprise orally administering the peptides disclosed herein to a non-human subject selected from the group consisting of a feline, a canine and an equine.

In an embodiment, the methods comprise administering the peptides disclosed herein topically to a human. In another embodiment, the methods comprise administering the peptides disclosed herein topically to a non-human subject. In yet another embodiment, the methods comprise administering the peptides disclosed herein topically to a non-human subject selected from the group consisting of a feline, a canine and an equine.

In another embodiment, the methods comprise administering the peptide of SEQ ID NO:2, or a pharmaceutically acceptable salt thereof, orally to a human. In another embodiment, the methods comprise administering the peptide of SEQ ID NO:2, or pharmaceutically acceptable salts thereof, orally to a non-human subject. In yet another embodiment, the methods comprise administering the peptide of SEQ ID NO:2, or pharmaceutically acceptable salts thereof, orally to a non-human subject selected from the group consisting of a feline, a canine and an equine.

In another embodiment, the methods comprise administering the peptide of SEQ ID NO:2, or a pharmaceutically acceptable salt thereof, topically to a human. In another embodiment, the methods comprise administering the peptide of SEQ ID NO:2, or pharmaceutically acceptable salts thereof, topically to a non-human subject. In yet another embodiment, the methods comprise administering the peptide of SEQ ID NO:2, or pharmaceutically acceptable salts thereof, topically to a non-human subject selected from the group consisting of a feline, a canine and an equine.

In another embodiment, the methods comprise administering the peptide of SEQ ID NO:3, or pharmaceutically acceptable salts thereof, orally to a non-human subject. In yet another embodiment, the methods comprise administering the peptide of SEQ ID NO:3, or pharmaceutically acceptable salts thereof, orally to a non-human subject selected from the group consisting of a feline, a canine and an equine.

In another embodiment, the methods comprise administering the peptide of SEQ ID NO:3, or pharmaceutically acceptable salts thereof, topically to a non-human subject. In yet another embodiment, the methods comprise administering the peptide of SEQ ID NO:3, or pharmaceutically acceptable salts thereof, topically to a non-human subject selected from the group consisting of a feline, a canine and an equine.

In another embodiment, the methods comprise administering the peptide of SEQ ID NO:4, or pharmaceutically acceptable salts thereof, orally to a non-human subject. In yet another embodiment, the methods comprise administering the peptide of SEQ ID NO:4, or pharmaceutically acceptable salts thereof, orally to a non-human subject selected from the group consisting of a feline, a canine and an equine.

In another embodiment, the methods comprise administering the peptide of SEQ ID NO:4, or pharmaceutically acceptable salts thereof, topically to a non-human subject. In yet another embodiment, the methods comprise administering the peptide of SEQ ID NO:4, or pharmaceutically acceptable salts thereof, topically to a non-human subject selected from the group consisting of a feline, a canine and an equine.

In another embodiment, the methods comprise administering the peptide of SEQ ID NO:5, or pharmaceutically acceptable salts thereof, orally to a non-human subject. In yet another embodiment, the methods comprise administering the peptide of SEQ ID NO:5, or pharmaceutically acceptable salts thereof, orally to a non-human subject selected from the group consisting of a feline, a canine and an equine.

In another embodiment, the methods comprise administering the peptide of SEQ ID NO:5, or pharmaceutically acceptable salts thereof, topically to a non-human subject. In yet another embodiment, the methods comprise administering the peptide of SEQ ID NO:5, or pharmaceutically acceptable salts thereof, topically to a non-human subject selected from the group consisting of a feline, a canine and an equine.

Illustrative examples of topical administration are described elsewhere herein. In an embodiment, the topical administration is transdermal.

In an embodiment disclosed herein, the peptides disclosed herein are administered to the subject as a controlled release dosage form, illustrative examples of which are described elsewhere herein. In an embodiment, the methods comprise administering the peptides disclosed herein to a human as a controlled release dosage form. In another embodiment, the methods comprise administering the peptides disclosed herein to a non-human subject as a controlled release dosage form. In yet another embodiment, the methods comprise administering the peptides disclosed herein as a controlled release dosage form to a non-human subject selected from the group consisting of a feline, a canine and an equine.

In another embodiment, the methods comprise administering the peptide of SEQ ID NO:2, or a pharmaceutically acceptable salt thereof, to a human as a controlled release dosage form. In another embodiment, the methods comprise administering the peptide of SEQ ID NO:2, or pharmaceutically acceptable salts thereof, to a non-human subject as a controlled release dosage form. In yet another embodiment, the methods comprise administering the peptide of SEQ ID NO:2, or pharmaceutically acceptable salts thereof, as a controlled release dosage form to a non-human subject selected from the group consisting of a feline, a canine and an equine.

In another embodiment, the methods comprise administering the peptide of SEQ ID NO:3, or pharmaceutically acceptable salts thereof, to a non-human subject as a controlled release dosage form. In yet another embodiment, the methods comprise administering the peptide of SEQ ID NO:3, or pharmaceutically acceptable salts thereof, as a controlled release dosage form to a non-human subject selected from the group consisting of a feline, a canine and an equine. In an embodiment, the controlled release dosage form is administered to the subject parenterally, suitable examples of which are described elsewhere herein.

In another embodiment, the methods comprise administering the peptide of SEQ ID NO:4, or pharmaceutically acceptable salts thereof, to a non-human subject as a controlled release dosage form. In yet another embodiment, the methods comprise administering the peptide of SEQ ID NO:4, or pharmaceutically acceptable salts thereof, as a controlled release dosage form to a non-human subject selected from the group consisting of a feline, a canine and an equine. In an embodiment, the controlled release dosage form is administered to the subject parenterally, suitable examples of which are described elsewhere herein.

In another embodiment, the methods comprise administering the peptide of SEQ ID NO:5, or pharmaceutically acceptable salts thereof, to a non-human subject as a controlled release dosage form. In yet another embodiment, the methods comprise administering the peptide of SEQ ID NO:5, or pharmaceutically acceptable salts thereof, as a controlled release dosage form to a non-human subject selected from the group consisting of a feline, a canine and an equine. In an embodiment, the controlled release dosage form is administered to the subject parenterally, suitable examples of which are described elsewhere herein.

As noted elsewhere herein, several (i.e., multiple) divided doses may be administered daily, weekly, monthly or other suitable time intervals, or the dose may be proportionally reduced as indicated by the exigencies of the situation. Where a course of multiple doses is required or otherwise desired, it may be beneficial to administer the peptides, as herein disclosed, via more than one route. For example, it may be desirable to administer a first dose parenterally (e.g., via intramuscular, intravenous; subcutaneous, epidural, intra-articular, intraperitoneal, intracisternal or intrathecal routes of administration) to induce a rapid or otherwise acute analgesic effect in a subject, followed by a subsequent (e.g., second, third, fourth, fifth, etc) dose administered enterally (e.g., orally or rectally) and/or topically (e.g., via transdermal or transmucosal routes of administration) to provide continuing availability of the active agent over an extended period subsequent to the acute phase of treatment. Alternatively, it may be desirable to administer a dose enterally (e.g., orally or rectally), followed by a subsequent (e.g., second, third, fourth, fifth, etc) dose administered parenterally (e.g., via intramuscular, intravenous; subcutaneous, epidural, intra-articular, intraperitoneal, intracisternal or intrathecal routes of administration) and/or topically (e.g., via transdermal or transmucosal routes of administration). Alternatively, it may be desirable to administer a dose topically (e.g., via transdermal or transmucosal routes of administration), followed by a subsequent (e.g., second, third, fourth, fifth, etc) dose administered parenterally (e.g., via intramuscular, intravenous; subcutaneous, epidural, intra-articular, intraperitoneal, intracisternal or intrathecal routes of administration) and/or enterally (e.g., orally or rectally).

The route of administration may suitably be selected on the basis of whether the neuropathic pain is localised or generalised, as discussed elsewhere herein. Alternatively, or in addition, the route of administration may suitably be selected having regard to factors such as the subject's general health, age, weight and tolerance (or a lack thereof) for given routes of administration (e.g., where there is a phobia of needles, an alternative route of administration may be selected, such as enteral and/or topical).

It is also to be understood that, where multiple routes of administration are desired, any combination of two or more routes of administration may be used in accordance with the methods disclosed herein. Illustrative examples of suitable combinations include, but are not limited to, (in order of administration), (a) parenteral-enteral; (b) parenteral-topical; (c) parenteral-enteral-topical; (d) parenteral-topical-enteral; (e) enteral-parenteral; (f) enteral-topical; (g) enteral-topical-parenteral; (h) enteral-parenteral-topical; (i) topical-parenteral; (j) topical-enteral; (k) topical-parenteral-enteral; (l) topical-enteral-parenteral; (m) parenteral-enteral-topical-parenteral; (n) parenteral-enteral-topical-enteral; etc.

In an embodiment, the methods comprise (i) parenterally administering to the subject the peptides disclosed herein, and (ii) non-parenterally (i.e, enterally or topically) administering to the subject the peptides disclosed herein, wherein the non-parenteral (enteral or topical) administration is subsequent to the parenteral administration. In an embodiment, the parenteral administration is selected from the group consisting of intramuscular, a subcutaneous and intravenous. In a further embodiment, the parenteral administration is subcutaneous. In an embodiment, the non-parenteral administration is oral.

In an embodiment, the methods disclosed herein comprise (i) parenterally administering to a human subject the peptides disclosed herein and (ii) orally administering to the human subject the peptides disclosed herein, wherein the oral administration is subsequent to the parenteral administration.

In another embodiment, the methods disclosed herein comprise (i) parenterally administering to a human subject the peptide of SEQ ID NO:2, or a pharmaceutically acceptable salt thereof, and (ii) orally administering to the human subject the peptide of SEQ ID NO:2, or a pharmaceutically acceptable salt thereof, wherein the oral administration is subsequent to the parenteral administration. In an embodiment, the parenteral administration is subcutaneous. In another embodiment, the parenteral administration is intrathecal.

In another embodiment, the methods disclosed herein comprise (i) parenterally administering to a human subject the peptide of SEQ ID NO:2, or a pharmaceutically acceptable salt thereof, and (ii) orally administering to the human subject the peptide of SEQ ID NO:2, or a pharmaceutically acceptable salt thereof, wherein the oral administration is subsequent to the parenteral administration. In an embodiment, the parenteral administration is subcutaneous. In another embodiment, the parenteral administration is intrathecal.

In an embodiment, the methods disclosed herein comprise (i) parenterally administering to a non-human subject the peptides disclosed herein, and (ii) orally administering to the non-human subject the peptides disclosed herein, wherein the oral administration is subsequent to the parenteral administration. In a further embodiment, the methods disclosed herein comprise (i) parenterally administering to a non-human subject the peptide of SEQ ID NO:4, or a pharmaceutically acceptable salt thereof, and (ii) orally administering to the non-human subject the peptide of SEQ ID NO:4, or a pharmaceutically acceptable salt thereof, wherein the oral administration is subsequent to the parenteral administration.

In an embodiment, the non-human subject is selected from the group consisting of a feline, a canine and an equine. In an embodiment, the parenteral administration is subcutaneous. In another embodiment, the parenteral administration is intrathecal.

In a further embodiment, the methods disclosed herein comprise (i) parenterally administering to a non-human subject the peptide of SEQ ID NO:5, or a pharmaceutically acceptable salt thereof, and (ii) orally administering to the non-human subject the peptide of SEQ ID NO:5, or a pharmaceutically acceptable salt thereof, wherein the oral administration is subsequent to the parenteral administration. In an embodiment, the non-human subject is selected from the group consisting of a feline, a canine and an equine. In an embodiment, the parenteral administration is subcutaneous. In another embodiment, the parenteral administration is intrathecal.

In a further embodiment, the methods disclosed herein comprise (i) parenterally administering to a human subject the peptides disclosed herein, and (ii) topically administering to the human subject the peptides disclosed herein, wherein the topical administration is subsequent to the parenteral administration.

In a further embodiment, the methods disclosed herein comprise (i) parenterally administering to a human subject the peptide of SEQ ID NO:2, or a pharmaceutically acceptable salt thereof, and (ii) topically administering to the human subject the peptide of SEQ ID NO:2, or a pharmaceutically acceptable salt thereof, wherein the topical administration is subsequent to the parenteral administration.

In a further embodiment, the methods disclosed herein comprise (i) parenterally administering to a human subject the peptide of SEQ ID NO:3, or a pharmaceutically acceptable salt thereof, and (ii) topically administering to the human subject the peptide of SEQ ID NO:3, or a pharmaceutically acceptable salt thereof, wherein the topical administration is subsequent to the parenteral administration.

In a further embodiment, the methods disclosed herein comprise (i) parenterally administering to a non-human subject the peptides disclosed herein, and (ii) topically administering to the non-human subject the peptides disclosed herein, wherein the topical administration is subsequent to the parenteral administration.

In a further embodiment, the methods disclosed herein comprise (i) parenterally administering to a non-human subject the peptide of SEQ ID NO:4, or a pharmaceutically acceptable salt thereof, and (ii) topically administering to the non-human subject the peptide of SEQ ID NO:4, or a pharmaceutically acceptable salt thereof, wherein the topical administration is subsequent to the parenteral administration.

In a further embodiment, the methods disclosed herein comprise (i) parenterally administering to a non-human subject the peptide of SEQ ID NO:5, or a pharmaceutically acceptable salt thereof, and (ii) topically administering to the non-human subject the peptide of SEQ ID NO:5, or a pharmaceutically acceptable salt thereof, wherein the topical administration is subsequent to the parenteral administration.

In an embodiment, the non-human subject is selected from the group consisting of a feline, a canine and an equine. In an embodiment, the parenteral route of administration is subcutaneous. In another embodiment, the topical route of administration is transdermal. In another embodiment, the parenteral administration is subcutaneous and the topical administration is transdermal.

Alternatively, or in addition, the peptides disclosed herein may suitably be administered as a controlled release dosage form. Thus, in an embodiment, the methods comprise (i) parenterally administering to the subject the peptides disclosed herein, and (ii) administering to the subject the peptides disclosed herein, as a controlled release dosage form, wherein the controlled release dosage form is administered subsequent to the parenteral administration. In another embodiment, the methods comprise (i) non-parenterally (enterally or topically) administering to the subject the peptides disclosed herein, and (ii) administering to the subject the peptides disclosed herein, as a controlled release dosage form, wherein the controlled release dosage form is administered to the subject subsequent to the non-parenteral administration. In yet another embodiment, the methods comprise (i) enterally administering to the subject the peptides disclosed herein, and (ii) administering to the subject the peptides disclosed herein, as a controlled release dosage form, wherein the controlled release dosage form is administered to the subject subsequent to the enteral administration. In yet another embodiment, the methods comprise (i) topically administering to the subject the peptides disclosed herein, and (ii) administering to the subject the peptides disclosed herein, as a controlled release dosage form, wherein the controlled release dosage form is administered to the subject subsequent to the topical administration. In a preferred embodiment, the controlled release dosage form is formulated for parenteral administration.

### Adjunct Therapy

The peptides disclosed herein may suitably be administered together, either sequentially or in combination (e.g., as an admixture), with one or more another active agents. It will be understood by persons skilled in the art that the nature of the other active agents will depend on the condition to be treated or prevented. For example, where the subject has cancer, the peptides disclosed herein may be administered to the subject together, either sequentially or in combination (e.g., as an admixture), with one or more chemotherapeutic agents, illustrative examples of which will be familiar to persons skilled in the art. Combination treatments of this nature can be advantageous by alleviating the neuropathic pain that is often associated with some chemotherapeutic agents, illustrative examples of which include cisplatin, carboplatin, oxaliplatin, vincristine, docetaxel, paclitaxel, izbepilone, bortezomib, thalidomide and lenalinomide. Thus, in an embodiment, the methods disclosed herein further comprise administering to the subject a therapeutically effective amount of a chemotherapeutic agent.

The peptides disclosed herein may also be suitably administered to the subject together, either sequentially or in combination (e.g., as an admixture), with one or more other analgesic agents capable of alleviating pain in the subject (i.e., other than prolactin, or a functional variant thereof, as described herein). Suitable analgesic agents will be familiar to persons skilled in the art, illustrative examples of which include analgesic agents capable of alleviating nociceptive pain, agents capable of alleviating neuropathic pain, or any combination thereof. Thus, in an embodiment, the methods disclosed herein further comprise administering to the subject a therapeutically effective amount of a second agent capable of alleviating pain in the subject, wherein the second agent is not prolactin, or a functional variant thereof, as described herein.

In another embodiment, the methods disclosed herein further comprise administering to the subject a therapeutically effective amount of a second agent capable of alleviating pain in the subject, wherein the second agent is not prolactin, or a functional variant thereof, as described herein.

In an embodiment, the second agent is capable of alleviating nociceptive pain in the subject. In another embodiment, the second agent is capable of alleviating neuropathic pain in the subject.

Suitable agents capable of alleviating nociceptive pain will be familiar to persons skilled in the art, illustrative examples of which include opiates such as morphine, codeine, dihydrocodeine, hydrocodone, acetyldihydrocodeine, oxycodone, oxymorphone and buprenorphine, and non-steroidal anti-inflammatory drugs (NSAIDs) such as aspirin, ibuprofen, naproxen, acetaminophen, diflunisal, salsalate, phenacetin, fenoprofen, ketoprofen, flurbiprofen, oxaprozin, loxoprofen, indomethacin, sulindac, etodolac, ketorolac, diclofenac, nabumetone, mefenamic acid, meclofenamic acid, flufenamic acid, tolfenamic acid, celecoxib, parecoxib, lumaricoxib, etoricoxib, firocoxib, rimesulide and licofelone. In an embodiment, the second agent capable of alleviating nociceptive pain is an opioid.

In other embodiments disclosed herein, the peptides disclosed herein are administered together, either sequentially or in combination (e.g., as an admixture), with another therapy to treat or alleviate neuropathic pain or the underlying condition that is causing the neuropathic pain. In some instances, the amount of the second neuropathic analgesic agent may be reduced when administration is together with the peptides disclosed herein. Illustrative examples of suitable agents capable of treating neuropathic pain include duloxetine, pregabalin, gabapentin, phenytoin, melatonin, carbamazepine, levocarnitine, capsaicin, tricyclic antidepressants such as amitryptiline and sodium channel blockers such as lidocaine.

### Pharmaceutical Compositions

The peptides disclosed herein may be formulated for administration to a subject as a neat chemical. However, in certain embodiments, it may be preferable to formulate the peptides disclosed herein as a pharmaceutical composition, including veterinary compositions. Thus, in another aspect disclosed herein, there is provided a pharmaceutical composition comprising prolactin, or a functional variant thereof, for use in the treatment and prevention of neuropathic pain in a subject, wherein the functional variant comprises a peptide of formula (I):

(SEQ ID NO: 1)
R¹-C-R-I-X₁-X₂-X₃-X₄-N-C-R² (I)

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent.

In an embodiment, the peptide is selected from the group consisting of

(SEQ ID NO: 2)
CRIIHNNNC,
(SEQ ID NO: 3)
CRIIHNNNCG,
(SEQ ID NO: 4)
CRIVYDSNC
and
(SEQ ID NO: 5)
CRIVYDSNCG.

In an embodiment, the peptide is CRIIHNNNC (SEQ ID NO:2). In an embodiment, the peptide is CRIIHNNNCG (SEQ ID NO:3). In an embodiment, the peptide is CRIVYDSNC (SEQ ID NO:4). In an embodiment, the peptide is

(SEQ ID NO: 5)
CRIVYDSNCG.

In an embodiment, the peptides disclosed herein are present in a therapeutically effective amount that, when administered to a subject, alleviates neuropathic pain in the subject, as described elsewhere herein.

In an embodiment, the composition further comprises a second agent capable of alleviating pain in the subject, wherein the second agent is not prolactin, or a functional variant thereof, as disclosed herein. In an embodiment, the second agent is capable of alleviating nociceptive pain in the subject, illustrative examples of which are described elsewhere herein. In another embodiment, the second agent is capable of alleviating neuropathic pain in the subject, illustrative examples of which are also described elsewhere herein. In an embodiment the second agent is an opioid.

In another aspect disclosed herein, there is provided a use of prolactin, or a functional variant thereof, in the manufacture of a medicament for the treatment and prevention of neuropathic pain in a subject, wherein the functional variant comprises a peptide of formula (I):

(SEQ ID NO: 1)
R¹-C-R-I-X₁-X₂-X₃-X₄-N-C-R² (I)

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent.

In an embodiment, the peptide is selected from the group consisting of CRIIHNNNC (SEQ ID NO:2), CRIIHNNNCG (SEQ ID NO:3), CRIVYDSNC (SEQ ID NO:4) and CRIVYDSNCG (SEQ ID NO:5). In an embodiment, the peptide is CRIIHNNNC (SEQ ID NO:2). In an embodiment, the peptide is CRIIHNNNCG (SEQ ID NO:3). In an embodiment, the peptide is CRIVYDSNC (SEQ ID NO:4). In an embodiment, the peptide is CRIVYDSNCG (SEQ ID NO:5).

In an embodiment, the peptides disclosed herein are formulated for administration to the subject in a therapeutically effective amount that alleviates neuropathic pain in the subject, as described elsewhere herein.

In an embodiment, the peptide is formulated for administration sequentially, or in combination, with a second agent capable of alleviating pain in the subject, wherein the second agent is not prolactin, or a functional variant thereof, as described herein. In an embodiment, the second agent is capable of alleviating nociceptive pain in the subject, illustrative examples of which are described elsewhere herein. In another embodiment, the second agent is capable of alleviating neuropathic pain in the subject, illustrative examples of which are also described elsewhere herein. In an embodiment, the second agent is an opioid.

As noted elsewhere herein, the peptides disclosed herein may be administered together, either sequentially or in combination (e.g., as an admixture), with one or more another active agents that will likely depend on the condition to be treated. For example, where the subject has cancer, the compositions disclosed herein may be formulated for administration together, either sequentially or in combination (e.g., as an admixture), with one or more chemotherapeutic agents, illustrative examples of which will be familiar to persons skilled in the art. Combination treatments of this nature can be advantageous by alleviating the neuropathic pain that is often associated with some chemotherapeutic agents, illustrative examples of which include cisplatin, carboplatin, oxaliplatin, vincristine, docetaxel, paclitaxel, izbepilone, bortezomib, thalidomide and lenalinomide.

The compositions disclosed herein may also be suitably formulated for administration to the subject together, either sequentially or in combination (e.g., as an admixture), with one or more other analgesic agents capable of alleviating pain in the subject described elsewhere herein (i.e., other than the prolactin, or functional variants thereof, as disclosed herein). In an embodiment, the compositions disclosed herein further comprise a second agent capable of alleviating pain in the subject, wherein the second agent is not prolactin, or a functional variant thereof, as described herein.

In an embodiment, the second agent is capable of alleviating nociceptive pain in the subject. In another embodiment, the second agent is capable of alleviating neuropathic pain in the subject.

Suitable agents capable of alleviating nociceptive pain will be familiar to persons skilled in the art, illustrative examples of which include opiates such as morphine, codeine, dihydrocodeine, hydrocodone, acetyldihydrocodeine, oxycodone, oxymorphone and buprenorphine, and non-steroidal anti-inflammatory drugs (NSAIDs) such as aspirin, ibuprofen, naproxen, acetaminophen, diflunisal, salsalate, phenacetin, fenoprofen, ketoprofen, flurbiprofen, oxaprozin, loxoprofen, indomethacin, sulindac, etodolac, ketorolac, diclofenac, nabumetone, mefenamic acid, meclofenamic acid, flufenamic acid, tolfenamic acid, celecoxib, parecoxib, lumaricoxib, etoricoxib, firocoxib, rimesulide and licofelone. In an embodiment, the second agent capable of alleviating nociceptive pain is an opioid.

In other embodiments disclosed herein, the compositions disclosed herein are formulated for administration together, either sequentially or in combination (e.g., as an admixture), with another therapy to treat or alleviate neuropathic pain or the underlying condition that is causing pain. In some instances, the amount of the second analgesic agent may be reduced when administration is together with the peptides disclosed herein. Illustrative examples of suitable agents capable of treating neuropathic pain include duloxetine, pregabalin, gabapentin, phenytoin, melatonin, carbamazepine, levocarnitine, capsaicin, tricyclic antidepressants such as amitryptiline and sodium channel blockers such as lidocaine.

In another aspect disclosed herein, there is provided a pharmaceutical composition comprising:

(i) prolactin, or a functional variant thereof, as described herein, wherein the functional variant comprises a peptide of formula (I):

(I)
(SEQ ID NO: 1)
R¹-C-R-I-X₁-X₂-X₃-X₄-N-C-R²

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent; and (ii) a second agent capable of alleviating pain in the subject, wherein the second agent is not prolactin, or a functional variant thereof, as described herein. In an embodiment, the second agent is capable of alleviating nociceptive pain in the subject, illustrative examples of which are described elsewhere herein. In another embodiment, the second agent is capable of alleviating neuropathic pain in the subject, illustrative examples of which are also described elsewhere herein. In an embodiment, the second agent is an opioid.

In an embodiment disclosed herein, the peptides disclosed herein are formulated as a composition comprising a pharmaceutically acceptable carrier, excipient or diluent. The carrier, excipient or diluent is generally considered “acceptable” where they are compatible with the other ingredients of the composition and give rise to little or no deleterious effects in the recipient.

In another aspect disclosed herein, there is provided an analgesic composition comprising prolactin, or a functional variant thereof, wherein the functional variant comprises a peptide of formula (I):

(I)
(SEQ ID NO: 1)
R¹-C-R-I-X₁-X₂-X₃-X₄-N-C-R²

wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent.

In an embodiment, the peptide is selected from the group consisting of

(SEQ ID NO: 2)
CRIIHNNNC,
(SEQ ID NO: 3)
CRIIHNNNCG,
(SEQ ID NO: 4)
CRIVYDSNC
and
(SEQ ID NO: 5)
CRIVYDSNCG.

In an embodiment, the peptide is CRIIHNNNC (SEQ ID NO:2). In an embodiment, the peptide is CRIIHNNNCG (SEQ ID NO:3). In an embodiment, the peptide is CRIVYDSNC (SEQ ID NO:4). In an embodiment, the peptide is CRIVYDSNCG (SEQ ID NO:5). In an embodiment, the analgesic composition further comprises a second agent capable of alleviating pain in the subject, as described elsewhere herein, wherein the second agent is not prolactin, or a functional variant thereof, as described herein. In an embodiment, the second agent is capable of alleviating nociceptive pain in the subject, illustrative examples of which are described elsewhere herein. In another embodiment, the second agent is capable of alleviating neuropathic pain in the subject, illustrative examples of which are also described elsewhere herein. In an embodiment, the second agent is an opioid.

In another aspect disclosed herein, there is provided a composition comprising a therapeutically effective amount of a peptide, or a pharmaceutically acceptable salt thereof, wherein the peptide consists, or consists essentially, of amino acid sequence CRIIHNNNC (SEQ ID NO:2) or amino acid sequence CRIIHNNNCG (SEQ ID NO:3).

In another aspect disclosed herein, there is provided a composition comprising a therapeutically effective amount of a peptide, or a pharmaceutically acceptable salt thereof, wherein the peptide consists, or consists essentially, of amino acid sequence CRIVYDSNC (SEQ ID NO:4) or CRIVYDSNCG (SEQ ID NO:5).

In an embodiment, the composition further comprises a pharmaceutically acceptable carrier, excipient or diluent, as described elsewhere herein. In an embodiment, the composition is formulated for oral administration.

Illustrative examples of suitable pharmaceutical formulations include those suitable for enteral or parenteral administration, illustrative examples of which are described elsewhere herein, including oral, rectal, buccal, sublingual, vaginal, nasal, topical (e.g., transdermal), intramuscular, subcutaneous, intravenous, epidural, intra-articular and intrathecal.

The peptides described herein may suitably be placed into the form of pharmaceutical compositions and unit dosages thereof to be employed as solids (e.g., tablets or filled capsules) or liquids (e.g., solutions, suspensions, emulsions, elixirs, or capsules filled with the same) for oral use, in the form of ointments, suppositories or enemas for rectal administration, in the form of sterile injectable solutions for parenteral use (e.g., intramuscular, subcutaneous, intravenous, epidural, intra-articular and intrathecal administration); or in the form of ointments, lotions, creams, gels, patches, sublingual strips or films, and the like for local (e.g., topical, buccal, sublingual, vaginal) administration. In an embodiment, the peptides of formula (I), or pharmaceutically acceptable salts thereof, are formulated for topical (e.g., transdermal) delivery. Suitable transdermal delivery systems will be familiar to persons skilled in the art, illustrative examples of which are described by Prausnitz and Langer (2008; Nature Biotechnol. 26(11):1261-1268), the contents of which are incorporated herein by reference. In another embodiment, the peptides of formula (I), or pharmaceutically acceptable salts thereof, are formulated for sublingual or buccal delivery. Suitable sublingual and buccal delivery systems will be familiar to persons skilled in the art, illustrative examples of which include dissolvable strips or films, as described by Bala et al. (2013; Int. J. Pharm. Investig. 3(2):67-76), the contents of which are incorporated herein by reference.

Suitable pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. The peptides disclosed herein can be formulated for administration in a wide variety of enteral, topical and/or parenteral dosage forms. Suitable dosage forms may comprise, as the active component, the peptides disclosed herein, as herein described.

As noted elsewhere herein, it may be desirable to elect a route of administration on the basis of whether the neuropathic pain is localized or generalised. For example, where the neuropathic pain is localized, it may be desirable to formulate the compositions disclosed herein for administration to the affected area or to an area immediately adjacent thereto. For instance, where the neuropathic pain is in a joint (e.g., neck, knee, elbow, shoulder or hip), the composition can be formulated for intra-articular administration into the affected joint. Alternatively, or in addition, the composition can be formulated for administration at, or substantially adjacent to, the affected joint. As another illustrative example, where the neuropathic pain is in the oral cavity (e.g., trigeminal neuropathic pain, atypical odontalgia (phantom tooth pain) or burning mouth syndrome), the composition can be formulated for administration via the oral mucosa (e.g., by buccal and/or sublingual administration).

Conversely, where the neuropathic pain is generalised or disseminated across multiple anatomical sites of a subject, it may be convenient to formulate the composition for enteral, topical and/or parenteral route of administration, as described elsewhere herein, with a view to distributing the active agents across the multiple anatomical sites affected by pain.

In an embodiment, the composition is formulated for oral administration to a human. In another embodiment, the composition is formulated for oral administration to a non-human subject. In yet another embodiment, the composition is formulated for oral administration to a non-human subject selected from the group consisting of a feline, a canine and an equine.

In another embodiment, the composition is formulated for parenteral administration to a human. In another embodiment, the composition is formulated for parenteral administration to a non-human subject. In yet another embodiment, the composition is formulated for parenteral administration to a non-human subject selected from the group consisting of a feline, a canine and an equine. In an embodiment, the parenteral administration is subcutaneous administration.

In another embodiment, the composition is formulated for topical administration to a human. In another embodiment, the composition is formulated for topical administration to a non-human subject. In yet another embodiment, the composition is formulated for topical administration to a non-human subject selected from the group consisting of a feline, a canine and an equine. In an embodiment, the topical administration is transdermal.

In another embodiment, the composition is formulated as a controlled release dosage form to be administered to a human. In another embodiment, the composition is formulated as a controlled release dosage form to be administered to a non-human subject. In yet another embodiment, the composition is formulated as a controlled release dosage form to be administered to a non-human subject selected from the group consisting of a feline, a canine and an equine. Illustrative examples of suitable controlled release dosage forms are described elsewhere herein.

For preparing pharmaceutical compositions of prolactin, or functional variants thereof, as herein described, pharmaceutically acceptable carriers can be either solid or liquid. Illustrative examples of solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material. In powders, the carrier may be a finely divided solid which is in a mixture with the finely divided active component. In tablets, the active component may be mixed with the carrier having the necessary binding capacity in suitable proportions and compacted in the shape and size desired.

In some embodiments, the powders and tablets contain from five or ten to about seventy percent of the active compound. Illustrative examples of suitable carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material, providing a capsule in which the active component, with or without carriers, is surrounded by a carrier. Similarly, cachets and lozenges are also envisaged herein. Tablets, powders, capsules, pills, cachets, and lozenges can be used as solid forms suitable for oral administration.

For preparing suppositories, a low melting wax, such as admixture of fatty acid glycerides or cocoa butter, is first melted and the active component is dispersed homogeneously therein, as by stirring. The molten homogenous mixture is then poured into convenient sized moulds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution.

The peptides disclosed herein may be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active compound(s) may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also contemplated herein are solid form preparations which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilizing agents, and the like.

For topical administration to the epidermis, the peptides of formula (I), or pharmaceutically acceptable salts thereof, as described herein, may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Formulations suitable for topical administration in the mouth include lozenges comprising active agent in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension. In the case of a spray, this may be achieved for example by means of a metering atomizing spray pump. To improve nasal delivery and retention the peptides used in the invention may be encapsulated with cyclodextrins, or formulated with their agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC) for example, dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively, or in addition, the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently, the powder carrier will form a gel in the nasal cavity. The powder composition may be presented in unit dose form for example in capsules or cartridges of, e.g., gelatin, or blister packs from which the powder may be administered by means of an inhaler.

In formulations intended for administration to the respiratory tract, including intranasal formulations, the peptide will generally have a small particle size for example of the order of 1 to 10 microns or less. Such a particle size may be obtained by means known in the art, for example by micronization.

When desired, formulations adapted to give controlled or sustained release of the active ingredient may be employed, as described elsewhere herein.

In an embodiment, the pharmaceutical preparations, as herein described, are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

In another aspect disclosed herein, there is provided a composition comprising a peptide of SEQ ID NO: 2 or SEQ ID NO:3, or a pharmaceutically acceptable salt thereof, as herein described, for use as a medicament.

In another aspect disclosed herein, there is provided a composition comprising a peptide of SEQ ID NO: 4 or SEQ ID NO:5, or a pharmaceutically acceptable salt thereof, as herein described, for use as a medicament.

In an embodiment, the compositions disclosed herein are formulated for oral administration to a human. In yet another embodiment, the compositions disclosed herein are formulated for oral administration to a non-human. In a further embodiment, the compositions disclosed herein are formulated for oral administration to a non-human selected from the group consisting of a feline, a canine and an equine.

In another embodiment, the peptides disclosed herein are formulated for oral administration to a human subject. In another embodiment, the peptides disclosed herein are formulated for oral administration to a non-human subject. In yet another embodiment, the peptides disclosed herein are formulated for oral administration to a non-human subject selected from the group consisting of a feline, a canine and an equine.

In another embodiment, the peptides disclosed herein are formulated for topical administration to a human subject. In yet another embodiment, the peptides disclosed herein are formulated for topical administration to a non-human subject. In another embodiment, the peptides disclosed herein are formulated for topical administration to a non-human subject selected from the group consisting of a feline, a canine and an equine. In an embodiment, the topical administration is transdermal.

In another embodiment, the peptides disclosed herein are formulated for administration to a human subject as a controlled release dosage form. In yet another embodiment, the peptides disclosed herein are formulated for administration to a non-human subject as a controlled release dosage form. In another embodiment, the peptides disclosed herein are formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of SEQ ID NO:2, or a pharmaceutically acceptable salt thereof, is formulated for oral administration to a human. In another embodiment, the peptide of SEQ ID NO:2, or a pharmaceutically acceptable salt thereof, is formulated for oral administration to a non-human subject. In yet another embodiment, the peptide of SEQ ID NO:2, or a pharmaceutically acceptable salt thereof, is formulated for oral administration to a non-human subject selected from the group consisting of a feline, a canine and an equine.

In another embodiment, the peptide of SEQ ID NO:2, or a pharmaceutically acceptable salt thereof, as disclosed herein, is formulated for topical administration to a human subject. In yet another embodiment, the peptide of SEQ ID NO:2, or a pharmaceutically acceptable salt thereof, as disclosed herein, is formulated for topical administration to a non-human subject. In another embodiment, the peptide of SEQ ID NO:2, or a pharmaceutically acceptable salt thereof, as disclosed herein, is formulated for topical administration to a non-human subject selected from the group consisting of a feline, a canine and an equine. In an embodiment, the topical administration is transdermal.

In another embodiment, the peptide of SEQ ID NO:2, or a pharmaceutically acceptable salt thereof, as disclosed herein, is formulated for administration to a human subject as a controlled release dosage form. In yet another embodiment, the peptide of SEQ ID NO:2, or a pharmaceutically acceptable salt thereof, as disclosed herein, is formulated for administration to a non-human subject as a controlled release dosage form. In another embodiment, the peptide of SEQ ID NO:2, or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of SEQ ID NO:3, or a pharmaceutically acceptable salt thereof, is formulated for oral administration to a non-human subject. In yet another embodiment, the peptide of SEQ ID NO:3, or a pharmaceutically acceptable salt thereof, is formulated for oral administration to a non-human subject selected from the group consisting of a feline, a canine and an equine.

In another embodiment, the peptide of SEQ ID NO:3, or a pharmaceutically acceptable salt thereof, is formulated for topical administration to a non-human subject. In yet another embodiment, the peptide of SEQ ID NO:3, or a pharmaceutically acceptable salt thereof, is formulated for topical administration to a non-human subject selected from the group consisting of a feline, a canine and an equine. In an embodiment, the topical administration is transdermal.

In another embodiment, the peptide of SEQ ID NO:3, or a pharmaceutically acceptable salt thereof, as disclosed herein, is formulated for administration to a human subject as a controlled release dosage form. In yet another embodiment, the peptide of SEQ ID NO:3, or a pharmaceutically acceptable salt thereof, as disclosed herein, is formulated for administration to a non-human subject as a controlled release dosage form. In another embodiment, the peptide of SEQ ID NO:3, or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of SEQ ID NO:3, or a pharmaceutically acceptable salt thereof, is formulated for oral administration to a non-human subject. In yet another embodiment, the peptide of SEQ ID NO:3, or a pharmaceutically acceptable salt thereof, is formulated for oral administration to a non-human subject selected from the group consisting of a feline, a canine and an equine.

In another embodiment, the peptide of SEQ ID NO:3, or a pharmaceutically acceptable salt thereof, is formulated for topical administration to a non-human subject. In yet another embodiment, the peptide of SEQ ID NO:3, or a pharmaceutically acceptable salt thereof, is formulated for topical administration to a non-human subject selected from the group consisting of a feline, a canine and an equine. In an embodiment, the topical administration is transdermal.

In another embodiment, the peptide of SEQ ID NO:3, or a pharmaceutically acceptable salt thereof, as disclosed herein, is formulated for administration to a human subject as a controlled release dosage form. In yet another embodiment, the peptide of SEQ ID NO:3, or a pharmaceutically acceptable salt thereof, as disclosed herein, is formulated for administration to a non-human subject as a controlled release dosage form. In another embodiment, the peptide of SEQ ID NO:3, or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of SEQ ID NO:4, or a pharmaceutically acceptable salt thereof, is formulated for oral administration to a non-human subject. In yet another embodiment, the peptide of SEQ ID NO:4, or a pharmaceutically acceptable salt thereof, is formulated for oral administration to a non-human subject selected from the group consisting of a feline, a canine and an equine.

In another embodiment, the peptide of SEQ ID NO:4, or a pharmaceutically acceptable salt thereof, is formulated for topical administration to a non-human subject. In yet another embodiment, the peptide of SEQ ID NO:4, or a pharmaceutically acceptable salt thereof, is formulated for topical administration to a non-human subject selected from the group consisting of a feline, a canine and an equine. In an embodiment, the topical administration is transdermal.

In another embodiment, the peptide of SEQ ID NO:4, or a pharmaceutically acceptable salt thereof, as disclosed herein, is formulated for administration to a human subject as a controlled release dosage form. In yet another embodiment, the peptide of SEQ ID NO:4, or a pharmaceutically acceptable salt thereof, as disclosed herein, is formulated for administration to a non-human subject as a controlled release dosage form. In another embodiment, the peptide of SEQ ID NO:4, or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

In another embodiment, the peptide of SEQ ID NO:5, or a pharmaceutically acceptable salt thereof, is formulated for oral administration to a non-human subject. In yet another embodiment, the peptide of SEQ ID NO:5, or a pharmaceutically acceptable salt thereof, is formulated for oral administration to a non-human subject selected from the group consisting of a feline, a canine and an equine.

In another embodiment, the peptide of SEQ ID NO:5, or a pharmaceutically acceptable salt thereof, is formulated for topical administration to a non-human subject. In yet another embodiment, the peptide of SEQ ID NO:5, or a pharmaceutically acceptable salt thereof, is formulated for topical administration to a non-human subject selected from the group consisting of a feline, a canine and an equine. In an embodiment, the topical administration is transdermal.

In another embodiment, the peptide of SEQ ID NO:5, or a pharmaceutically acceptable salt thereof, as disclosed herein, is formulated for administration to a human subject as a controlled release dosage form. In yet another embodiment, the peptide of SEQ ID NO:5, or a pharmaceutically acceptable salt thereof, as disclosed herein, is formulated for administration to a non-human subject as a controlled release dosage form. In another embodiment, the peptide of SEQ ID NO:5, or a pharmaceutically acceptable salt thereof, as disclosed herein, is formulated for administration to a non-human subject as a controlled release dosage form, wherein the non-human subject is selected from the group consisting of a feline, a canine and an equine. In an embodiment, the controlled release dosage form is formulated for parenteral administration.

As noted elsewhere herein, several (i.e., multiple) divided doses may be administered daily, weekly, monthly or other suitable time intervals, or the dose may be proportionally reduced as indicated by the exigencies of the situation. Where a course of multiple doses is required or otherwise desired, the compositions disclosed herein can be suitably formulated for administration via said multiple routes. For example, it may be desirable to administer a first dose parenterally (e.g., intramuscular, intravenously; subcutaneously, etc) to induce a rapid or otherwise acute analgesic effect in a subject, followed by a subsequent (e.g., second, third, fourth, fifth, etc) dose administered non-parenterally (e.g., enterally and/or topically) to provide continuing availability of the active agent over an extended period subsequent to the acute phase of treatment. Thus, in an embodiment, the peptides and compositions, as disclosed herein, are formulated for parenteral administration to the subject as a first dose (i.e., as a parenteral dosage form) and formulated for non-parenteral administration to the subject after the first dose (e.g., as an enteral and/or topical dosage form). In an embodiment, the parenteral administration is selected from the group consisting of intramuscular, subcutaneous and intravenous. In a further embodiment, the parenteral administration is subcutaneous.

In another embodiment, the enteral administration is oral administration. Thus, in an embodiment, the peptides and compositions, as disclosed herein, are formulated for parenteral administration to the subject as a first dose and formulated for oral administration to the subject after the first dose (i.e., as an oral dosage form).

In another embodiment, the enteral administration is topical administration. Thus, in an embodiment, the peptides and compositions, as disclosed herein, are formulated for parenteral administration to the subject as a first dose and formulated for topical administration to the subject after the first dose (i.e., as an oral dosage form). In an embodiment, the topical administration is transdermal administration.

In another embodiment, it may be desirable to administer a first dose parenterally (e.g., intramuscular, intravenously; subcutaneously, etc) to induce a rapid or otherwise acute analgesic effect in a subject, followed by a subsequent (e.g., second, third, fourth, fifth, etc) administration of a controlled release dosage form, as described elsewhere herein, to provide a controlled release of the active agent over an extended period subsequent to the acute phase of treatment. Thus, in another embodiment, the peptides and compositions, as disclosed herein, are formulated for parenteral administration to the subject as a first dose and formulated as a controlled release dosage form to be administered to the subject after the first dose. In an embodiment, the controlled release dosage form is formulated for parenteral administration.

It may also be desirable to administer a first dose enterally (e.g., orally or rectally), followed by a subsequent (e.g., second, third, fourth, fifth, etc) dose administered topically (e.g., transdermally). Thus, in an embodiment, the peptides and compositions, as disclosed herein, are formulated for enteral administration to the subject as a first dose (i.e., as an enteral dosage form; oral or rectal) and formulated for topical administration to the subject after the first dose (e.g., as a transdermal or transmucosal dosage form). In another embodiment, the peptides and compositions, as disclosed herein, are formulated for topical administration selected from the group consisting of transdermal and transmucosal administration. In a further embodiment, the peptides and compositions, as disclosed herein, are formulated for transdermal administration.

In yet another embodiment, it may be desirable to administer the peptides or compositions, as disclosed herein, enterally (e.g., orally or rectally) as a first dose, followed by a subsequent (e.g., second, third, fourth, fifth, etc) dose as a controlled release dosage form, as described elsewhere herein. Thus, in an embodiment, the peptides and compositions, as disclosed herein, are formulated for administration as a first dose enterally and formulated for administration as a controlled release dosage form, wherein the controlled release dosage form is formulated for administration subsequent to the first dose. In an embodiment, the enteral dose is formulated for oral administration. In another embodiment, the controlled release dosage form is formulated for parenteral administration.

In an embodiment, it may be desirable to administer the peptides or compositions, as disclosed herein, topically (e.g., orally or rectally) as a first dose, followed by a subsequent (e.g., second, third, fourth, fifth, etc) dose as a controlled release dosage form, as described elsewhere herein. Thus, in an embodiment, the peptides and compositions, as disclosed herein, are formulated for topical administration as a first dose and formulated for administration as a controlled release dosage form, wherein the controlled release dosage form is formulated for administration subsequent to the first topical dose. In an embodiment, the topical dose is formulated for transdermal administration. In another embodiment, the controlled release dosage form is formulated for parenteral administration.

The invention will now be described with reference to the following Examples which illustrate some preferred aspects of the present invention. However, it is to be understood that the particularity of the following description of the invention is not to supersede the generality of the preceding description of the invention.

### EXAMPLES

Peptides comprising the amino acid sequence of SEQ ID NOs:2, 3, 4 and 5 (LAT7771, LAT7772, LAT7773 and LAT7774, respectively) were synthesized by Auspep (Victoria, Australia) using solid phase synthesis and Fmoc protection strategy. LAT7771, LAT7772, LAT7773 and LAT7774 are soluble in distilled water, and made up as concentrated stocks and stored frozen until the day of the experiment, when they were diluted to the required test concentrations in artificial cerebrospinal fluid (aCSF).

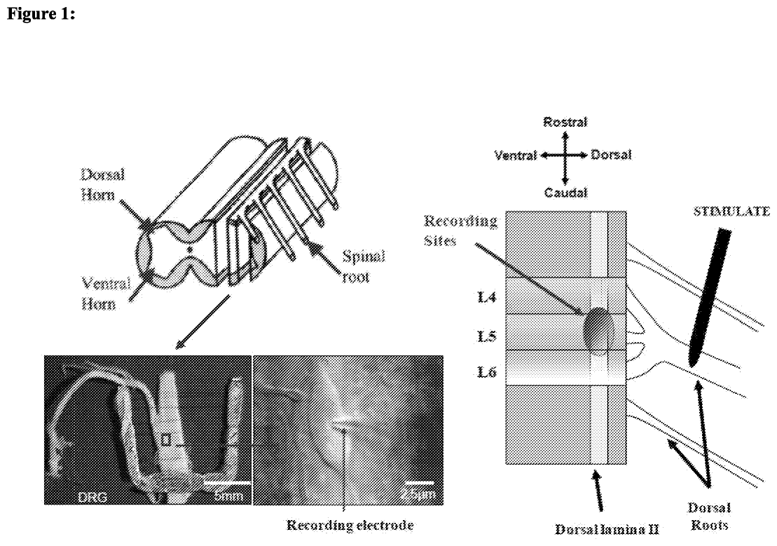

An in vitro spinal cord slice with intact dorsal root afferents combined with single-cell whole-cell patch clamp electrophysiological recording techniques was used to assess the electrophysiological properties of the peptides having the amino acid sequence of SEQ ID NO:2, 3, 4 and 5. A schematic diagram of the experimental preparation is shown in FIG. 1.

The spinal nerve ligation model (Chung model) was first reported by Kim and Chung (1992; Pain, 50(3):355-63) and involves a single tight ligation of the L5 spinal nerve. The model shows characteristic features of neuropathic pain symptoms/signs such as: mechanical allodynia, mechanical and thermal hyperalgesia and spontaneous pain that mimics the symptoms/signs observed in clinical patients. This model has been used as a “gold standard” model for assessing the efficacy of novel compounds targeting neuropathic pain.

Adult male Sprague-Dawley rats, 8-9 weeks old, weighing 220-250 g at the time of surgery, were purchased from Charles River UK Ltd. The animals were housed in groups of 4 in an air-conditioned room on a 12-hour light/dark cycle. Food and water were available ad libitum. They were allowed to acclimatise to the experimental environment for three days by leaving them on a raised metal mesh for at least 40 min. The baseline paw withdrawal threshold (PWT) was examined using a series of graduated von Frey hairs for 3 consecutive days before surgery and re-assessed on the 6th to 8th day after surgery and on the 12th to 14th day after surgery before drug dosing. Each rat was anaesthetized with 5% isoflurane mixed with oxygen (2L per min) followed by an intramuscular (i.m.) injection of ketamine 90 mg/kg plus xylazine 10 mg/kg. The back was shaved and sterilized with povidone-iodine. The animal was placed in a prone position and a para-medial incision was made on the skin covering the L4-6 level. The L5 spinal nerve was carefully isolated and tightly ligated with 6/0 silk suture. The wound was then closed in layers after a complete hemostasis. A single dose of antibiotics (Amoxipen, 15 mg/rat, i.p.) was routinely given for prevention of infection after surgery. The animals were placed in a temperature-controlled recovery chamber until fully awake before being returned to their home cages.

Chung model rats, aged 8 to 12 weeks, were housed in an air-conditioned room on a 12 hour light/dark cycle with food and water available ad libitum. The rats were terminally anaesthetized using isofluorane and decapitated. The vertebral column, rib cage and surrounding tissues were rapidly removed and pinned under ice-cold (<4° C.), high sucrose-containing artificial cerebrospinal fluid (aCSF) comprising: 127 mM sucrose, 1.9 mM KCl, 1.2 mM KH2PO₄, 0.24 mM CaCl₂, 3.9 mM MgCl₂, 26 mM NaHCO₃, 10 mM D-glucose and 0.5 mM ascorbic acid. A laminectomy was performed and the spinal cord and associated roots gently dissected and teased out of the spinal column and surrounding tissues. Dura and pia mater and ventral roots were subsequently removed with fine forceps and the spinal cord hemisected. Care was taken to ensure dorsal root inputs to the spinal cord were maintained. The hemisected spinal cord-dorsal root preparations were secured to a tissue slicer and spinal cord slices (400 to 450 μm thick) with dorsal roots attached were cut in chilled (<4° C.) high sucrose aCSF using a Leica VT1000s microtome.

Slices were transferred to a small beaker containing ice cold aCSF with 127 mM NaCl, 1.9 mM KCl, 1.2 mM KH₂PO₄, 1.3 mM MgCl₂, 2.4 mM CaCl₂, 26 mM NaHCO3 and 10 mM D-glucose, and rapidly warmed to 35° C. ±1° C. in a temperature-controlled water bath over a 20 minute period, then subsequently removed and maintained at room temperature (22° C. ±2° C.) prior to electrophysiological recording. Electrophysiological recording was performed in aCSF comprising 127 mM NaCl, 1.9 mM KCl, 1.2 mM KH₂PO₄, 1.3 mM MgCl₂, 2.4 mM CaCl₂, 26 mM NaHCO₃ and 10 mM D-glucose.

Whole-cell recordings were performed at 34-35° C. from Lamina I or II neurones in the dorsal horn of the spinal cord slices using Axopatch 1D and/or Multiclamp 700B amplifies and using the “blind” version of the patch-clamp technique.

Patch pipettes were pulled from thin-walled borosilicate glass with resistances of between 3 and 8 MS2 when filled with intracellular solution (140 mM potassium gluconate, 10 mM KCl, 1 mM EGTA-Na, 10 mM HEPES, 4 mM Na₂ATP, 0.3 mM Na-GTP). The peptides of SEQ ID NOs:2, 3, 4 or 5 (LAT7771, LAT7772, LAT7773 or LAT7774) were diluted in the tissue bath at a concentration of 5 μM.

A concentric bipolar stimulating electrode was placed appropriately either on the dorsal root or dorsal root entry zone to evoke postsynaptic potentials/currents in recorded cells (see FIG. 1). Low frequency single shocks were delivered to evoke excitatory postsynaptic currents (EPSCs) and potentials (EPSPs) in voltage- and current-clamp, respectively (0.1-0.8 ms pulse width, 2-35 V, 0.16-0.33 Hz). EPSPs/EPSCs were confirmed as monosynaptic on the basis of being characterised by a constant latency and rise-time with few failures.

Recordings were monitored on an oscilloscope and stored on digital audio tapes for later off-line analysis. In addition, data were low-pass filtered at 2-5 kHz, (1 kHz for voltage-clamp data), digitized at 2-10 kHz and stored on a PC running pCLAMP 10 data acquisition software (Axon Instruments). Analysis of electrophysiological data was carried out using Clampfit 10 software.

This study was undertaken to assess the effect of the peptides of SEQ ID NOs:2, 3, 4 or 5 (LAT7771, LAT7772, LAT7773 or LAT7774) on spinal cord dorsal horn neurones in a chronic nerve constriction model using CCI rats, which were prepared as outlined above.

### Example 1

### The Effects of LAT7771 on Dorsal Horn Neurones

### (i) Effect of LAT 7771 on Membrane Properties of Dorsal Horn Neurones

The effects of LAT7771 (5 μM) were investigated on 11 dorsal horn neurones in spinal cord slices prepared from Chung models of neuropathic pain, recorded ipsilateral to the site of injury. In these neurones, LAT7771 induced a small membrane depolarisation in 8 neurones, ranging between 1.7 and 10 mV, and had little effect on a further 3 dorsal horn neurones.

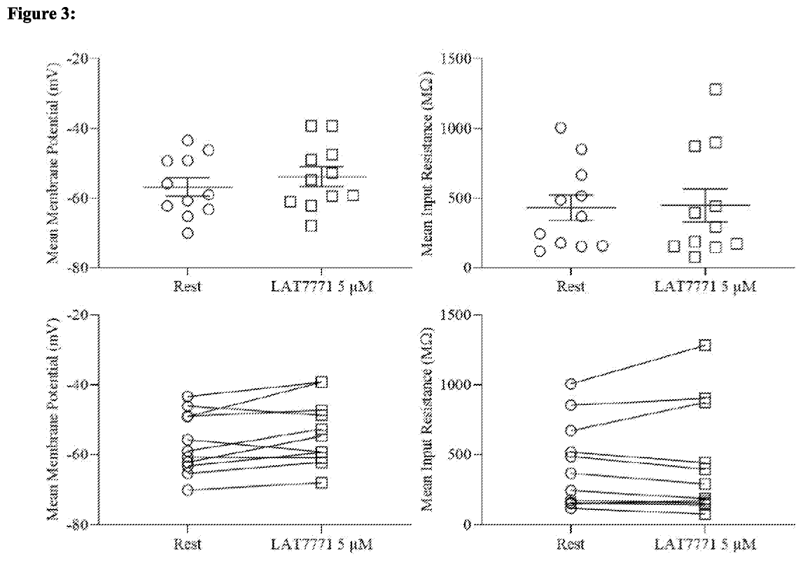

Data for all cells (n=11) showed LAT7771 induced membrane depolarisation from a mean resting membrane potential of −56.8±2.6 mV to a new steady-state resting potential of −53.8±2.8 mV, amounting to a 2.9±1.2 mV change in membrane potential (n=11, P<0.05, Table 1, FIG. 2, 3). LAT7771-induced responses were associated overall with little change in neuronal input resistance. Neuronal input resistance was increased from a mean control resting level of 431.8±91.8 MS2 to 447.6±119.4 MS2 (n=11, P=0.671) in the presence of LAT7771, amounting to a 15.8±36.1 MS2 increase in neuronal input resistance. FIGS. 2 and 3 summarise the effects of LAT7771 on membrane properties of dorsal horn neurones (see also Table 1).

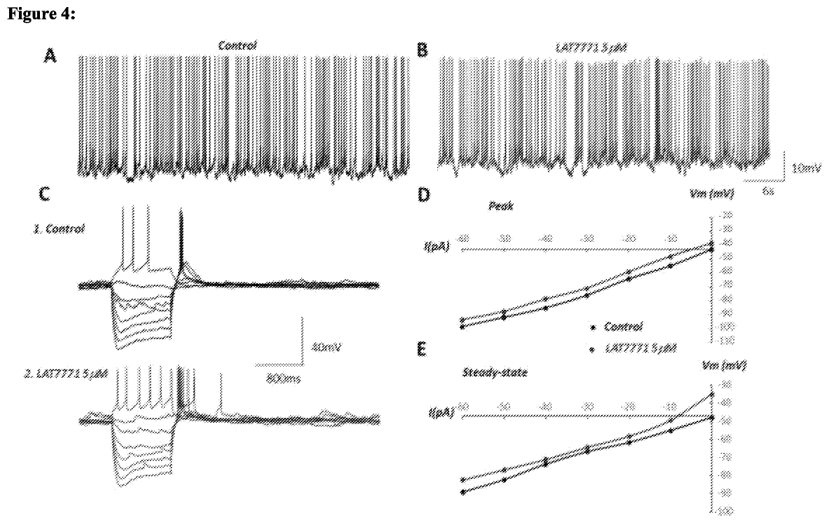

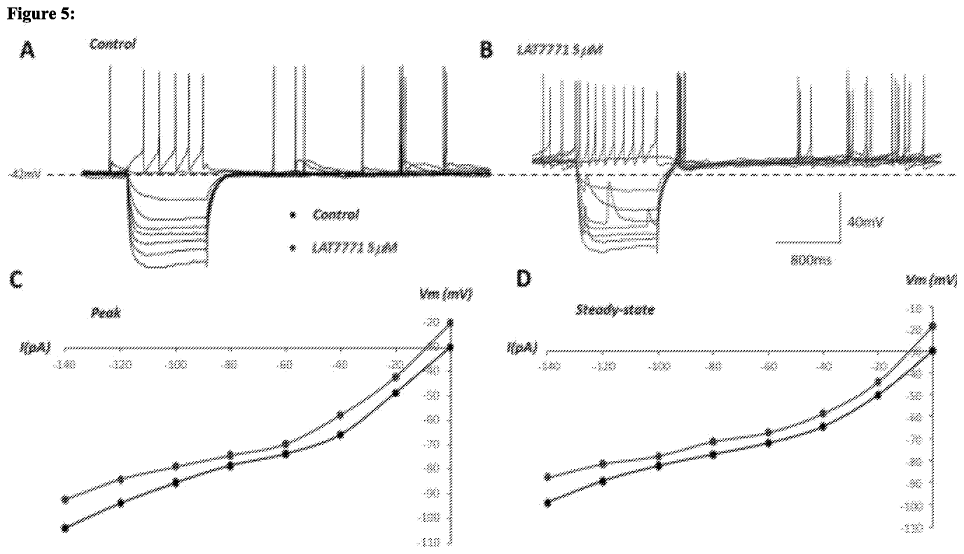

The effects of LAT7771 were further investigated on postsynaptic membrane properties. Whilst shifts in membrane potential were observed, these effects were often not associated with notable changes in neuronal input resistance with VI relations most commonly revealing parallel shifts with no obvious reversal potential (see FIGS. 4 and 5). This may reflect activity at electrogenic ion exchangers or pumps or a combination of ion channels being activated to effectively negate changes in conductance, for example simultaneous activation and inhibition of two species of ionic conductance. However, in two neurones, with little change in the resting potential, an enhancement or increase in the strength of inward rectification was observed (see FIG. 5). In four other neurones, changes in membrane potential were associated with reversal potentials of −55 mV, −55 mV, −45 mV and −40 mV, respectively reflecting activation of a chloride conductance or non-selective cation channels.

### (ii) The Effect of LAT 7771 on Dorsal Root Afferent-Mediated Synaptic Inputs

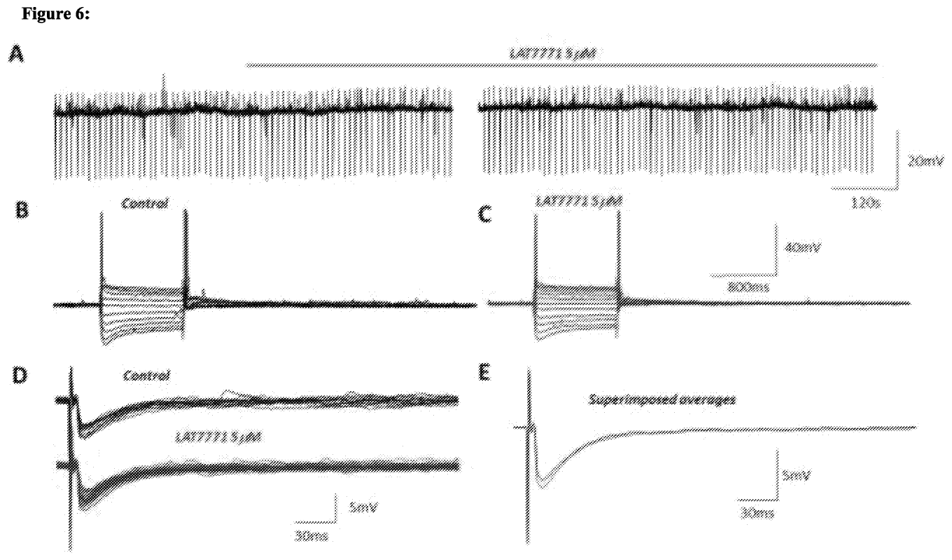

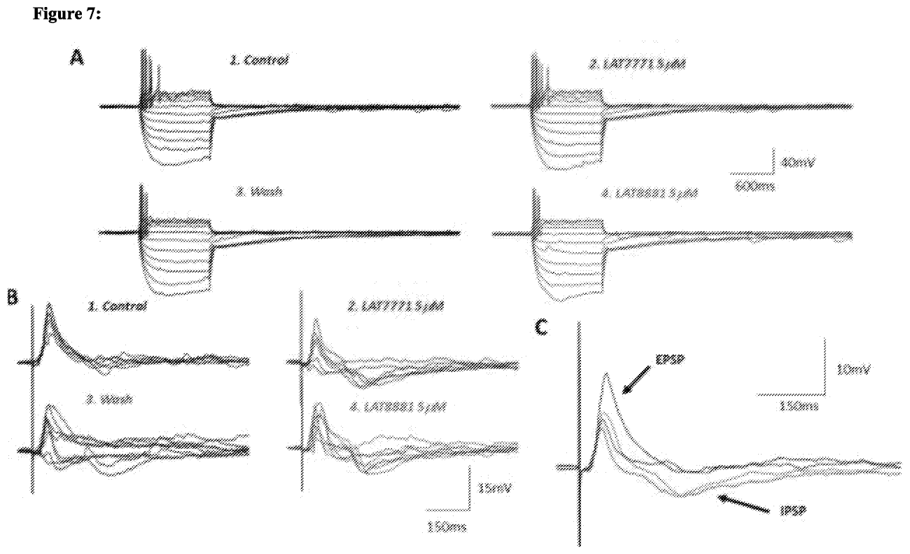

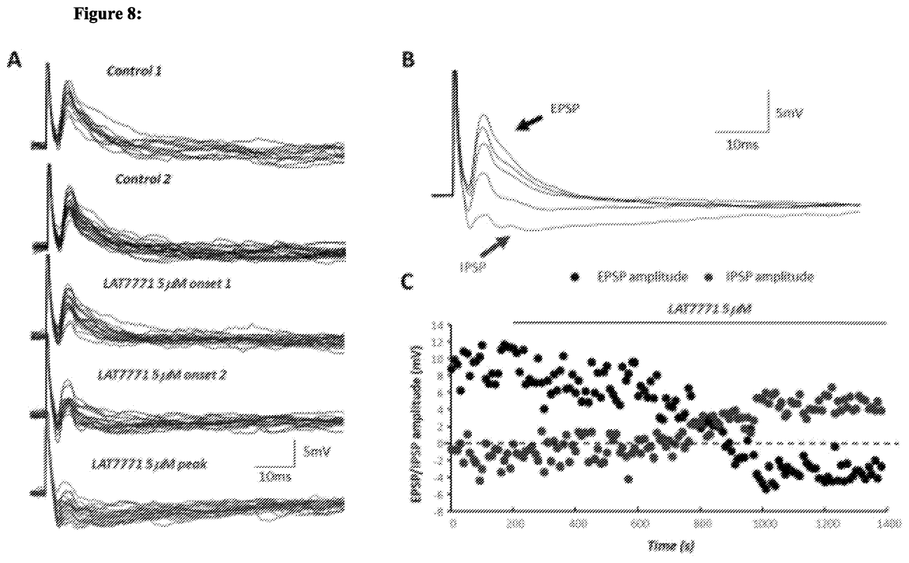

The effects of LAT7771 were also tested on dorsal root afferent-mediated synaptic inputs. Here, LAT7771 showed some remarkable effects on excitatory postsynaptic potentials (EPSPs) and inhibitory postsynaptic potentials (IPSPs). In four neurones, dorsal root evoked EPSPs at a frequency of 0.1 Hz were reduced in the presence of LAT7771. In two of these neurones, suppression of EPSPs uncovered or activated IPSPs such that inhibitory synaptic transmission to the dorsal horn was promoted and superseded excitatory inputs (see FIGS. 7 and 8). In one neurone, this effect of LAT7771 was partly mimicked by LAT8881, the human growth hormone derived compound of PCT/AU2019/050020, which has been shown to have analgesic properties (see FIG. 7). In a further neurone, IPSPs evoked by stimulation of dorsal roots in the absence of excitatory inputs, were also enhanced in LAT7771 (see FIG. 6). These effects on dorsal root afferent-mediated inputs were observed in the absence of significant effects on postsynaptic cells suggesting a presynaptic site of action (see FIGS. 6 and 7).

TABLE 1
Summary data of changes in membrane potential and input resistance
associated with LAT7771-induced responses in dorsal horn
neurones from Chung models of neuropathic pain.

| | Rest | LAT7771 5 μM | ΔVm |
|---|---|---|---|
| | Membrane Potential (mV) | | |
| Mean | −56.8 | −53.8 | −2.9 |
| SEM | 2.6 | 2.8 | 1.2 |
| n | 11 | 11 | 11 |
| P | | 0.039 | |
| | Normalised Membrane Potential | | |
| Mean | 100 | 94.8 | 5.2 |
| SEM | | 2.4 | 2.4 |
| n | 11 | 11 | 11 |
| P | | 0.052 | |
| | Control | LAT7771 5 μM | ΔIR |
| | Input Resistance (MΩ) | | |
| Mean | 431.8 | 447.6 | −15.8 |
| SEM | 91.8 | 119.4 | 36.1 |
| n | 11 | 11 | 11 |
| P | | 0.671 | |
| | Normalised Input Resistance | | |
| Mean | 100 | 94.8 | 5.2 |
| SEM | | 6.4 | 6.4 |
| n | 11 | 11 | 11 |
| P | | 0.439 | |

### Example 2

### The Effects of LAT7772 on Dorsal Horn Neurones

### (i) Effect of LAT 7772 on Membrane Properties of Dorsal Horn Neurones

The effects of LAT7772 (5 μM) were investigated on 5 dorsal horn neurones in spinal cord slices prepared from Chung models of neuropathic pain, recorded ipsilateral to the site of injury. In these neurones, LAT7772 overall had little effect on membrane potential, inducing a small membrane depolarisation in 2 neurones, a substantial membrane depolarisation in one neurone amounting to 12.7 mV, and a membrane hyperpolarisation with associated fall in input resistance in the remaining two neurones amounting to 3.5 and 9.9 mV. LAT7772 induced a decrease in neuronal input resistance in all 5 neurones.

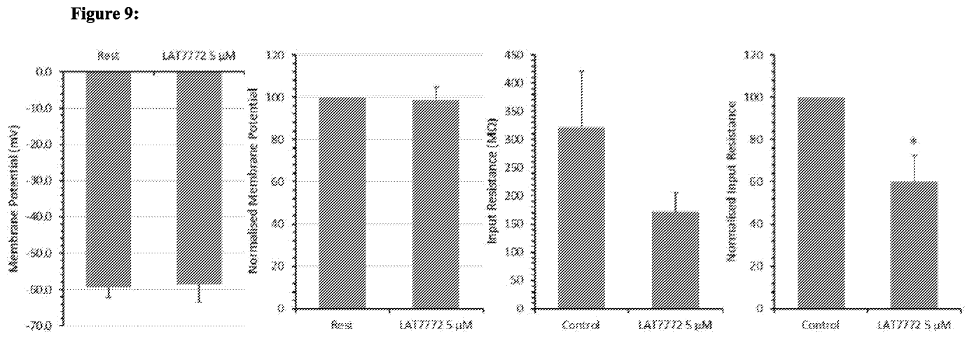

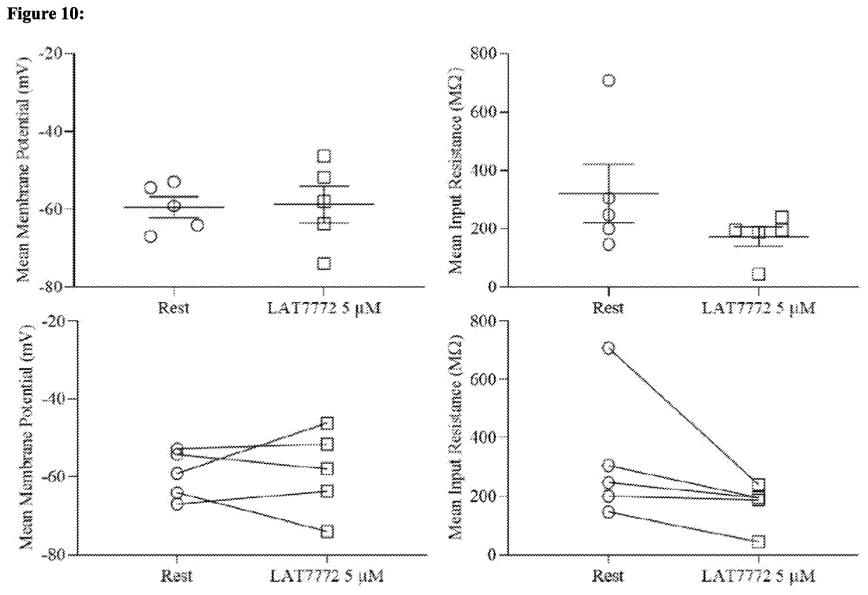

Data for all cells (n=5) showed LAT7772 induced little change in membrane, inducing depolarisation from a mean resting membrane potential of −59.5±2.7 mV to a new steady-state resting potential of −58.7±4.8 mV, amounting to a 0.8±3.7 mV change in membrane potential (n=5, P=0.849, Table 2, FIG. 9, 10). LAT7772-induced responses were associated overall with a significant decrease in neuronal input resistance from a mean control resting level of 321.4±100.1 MS2 to 172.3±33.2 MS2 (n=5, P=0.142) in the presence of LAT7772, amounting to a 149.1±81.7 MS2 (60.1±12.3% of control) fall in neuronal input resistance. FIGS. 9 and 10 summarise the effects of LAT7772 on membrane properties of dorsal horn neurones (see also Table 2).

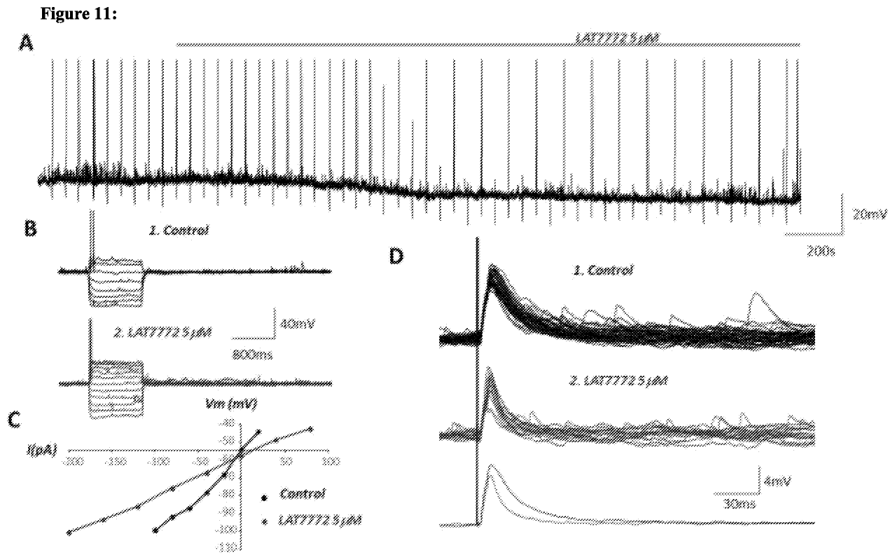

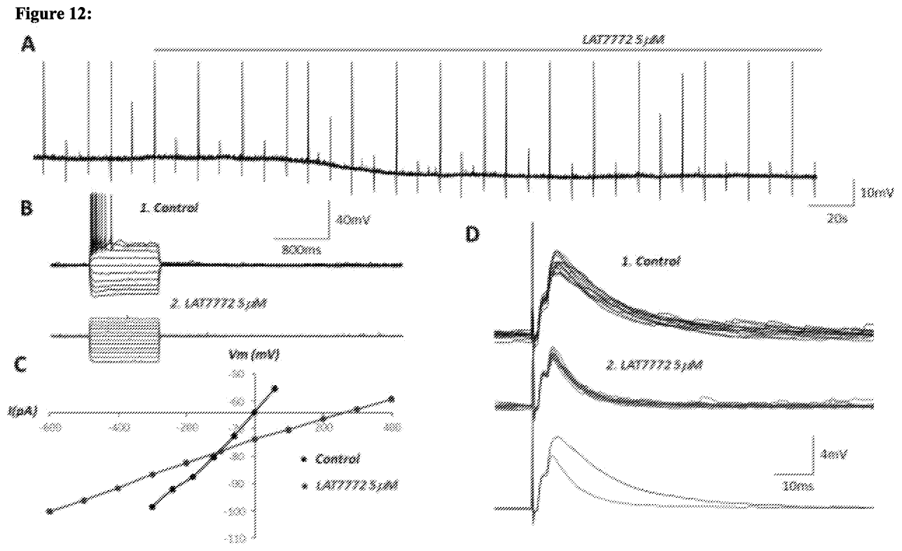

LAT7772 induced a clear membrane hyperpolarisation and inhibition of activity in two cells (see FIGS. 11 and 12). The reversal potential associated with LAT7772-induced inhibition were around −78 mV and −55 mV, mid-way between potassium and chloride ion reversal potentials and chloride ions alone, respectively. LAT7772-induced depolarisation in three cells was associated with reversal potentials of −100 mV, −60 mV and −60 mV, respectively. These data suggest LAT7772 induced depolarisation via block of potassium conductances (in the case of the reversal potential predicted around −100 mV) while those reversing around −60 mV suggest engagement of chloride-selective conductances.

### (ii) The Effect of LAT 7772 on Dorsal Root Afferent-Mediated Synaptic Inputs

The effects of LAT7772 were also tested on dorsal root afferent-mediated synaptic inputs. LAT7772 suppressed dorsal root-evoked excitatory posts-synaptic potentials (EPSPs) in all neurones tested (FIGS. 11, 12 and 13). As LAT7772 effects were often associated with a reduction in postsynaptic input resistance; at present it is not possible to know for certain if these effects on dorsal root afferent-mediated EPSPs are direct on presynaptic terminals or reflect indirect consequences of changes to the postsynaptic cell.

TABLE 2
Summary data of changes in membrane potential and input resistance
associated with LAT7772-induced responses in dorsal horn
neurones from Chung models of neuropathic pain.

| | Rest | LAT7772 5 μM | ΔVm |
|---|---|---|---|
| | Membrane Potential (mV) | | |
| Mean | −59.5 | −58.7 | −0.8 |
| SEM | 2.7 | 4.8 | 3.7 |
| n | 5 | 5 | 5 |
| P | | 0.849 | |
| | Normalised Membrane Potential | | |
| Mean | 100 | 98.6 | 1.4 |
| SEM | | 6.2 | 6.2 |
| n | 5 | 5 | 5 |
| P | | 0.835 | |
| | Control | LAT7772 5 μM | ΔIR |
| | Input Resistance (MΩ) | | |
| Mean | 321.4 | 172.3 | 149.1 |
| SEM | 100.1 | 33.2 | 81.7 |
| n | 5 | 5 | 5 |
| P | | 0.142 | |
| | Normalised Input Resistance | | |
| Mean | 100 | 60.1 | 39.9 |
| SEM | | 12.3 | 12.3 |
| n | 5 | 5 | 5 |
| P | | 0.032 | |

### Example 3

### The Effects of LAT7773 on Dorsal Horn Neurones

### (i) Effect of LAT 7773 on Membrane Properties of Dorsal Horn Neurones

The effects of LAT7773 (5 μM) were investigated on 9 dorsal horn neurones in spinal cord slices prepared from Chung models of neuropathic pain, recorded ipsilateral to the site of injury. In these neurones, LAT7773 induced membrane depolarisation in all but 1 neurone, ranging between 1.5 and 25.6 mV and induced a 7.6 mV membrane hyperpolarisation in the remaining neurone. LAT7773 had variable effects on input resistance inducing a decrease in 4 neurones and an increase in 5 neurones.

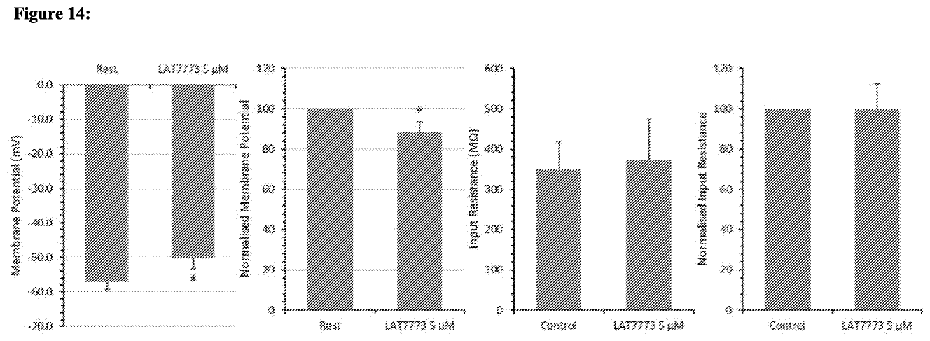

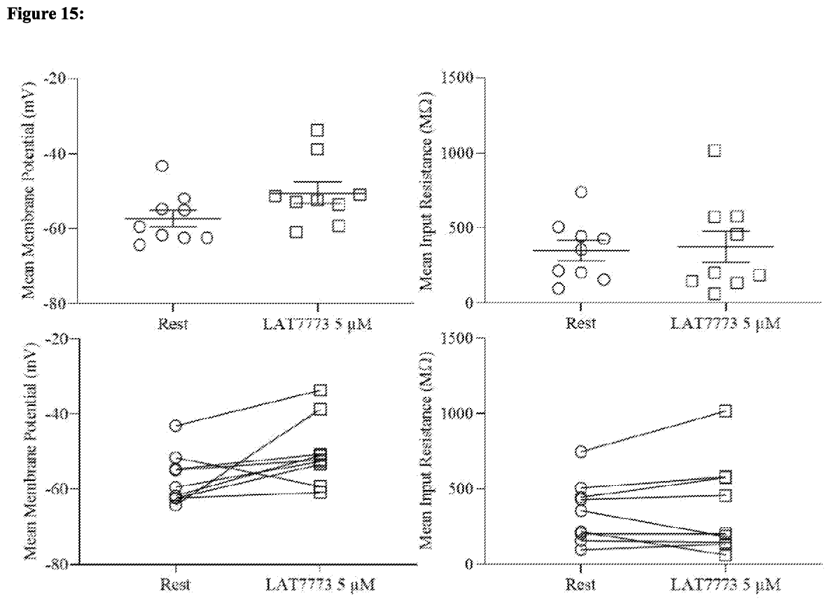

Data for all cells (n=9) showed LAT7773 induced membrane depolarisation from a mean resting membrane potential of −57.2±2.3 mV to a new steady-state resting potential of −50.3±3.0 mV, amounting to a 6.9±3.0 mV change in membrane potential (n=9, P<0.05, Table 3). LAT7773-induced responses were associated overall with a small increase in neuronal input resistance although effects of the compound were variable. Neuronal input resistance was increased from a mean control resting level of 350.6±68.1 MΩ to 373.5±103.6 MΩ (n=9, P=0.625) in the presence of LAT7773, amounting to a 22.9±45.1 MΩ increase in neuronal input resistance. FIGS. 14 and 15 summarises the effects of LAT7773 on membrane properties of dorsal horn neurones (see also Table 3).

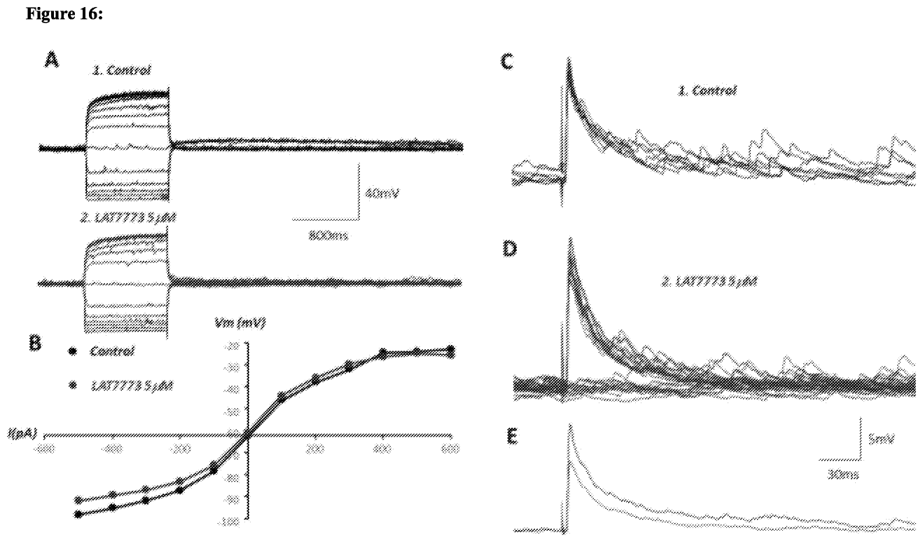

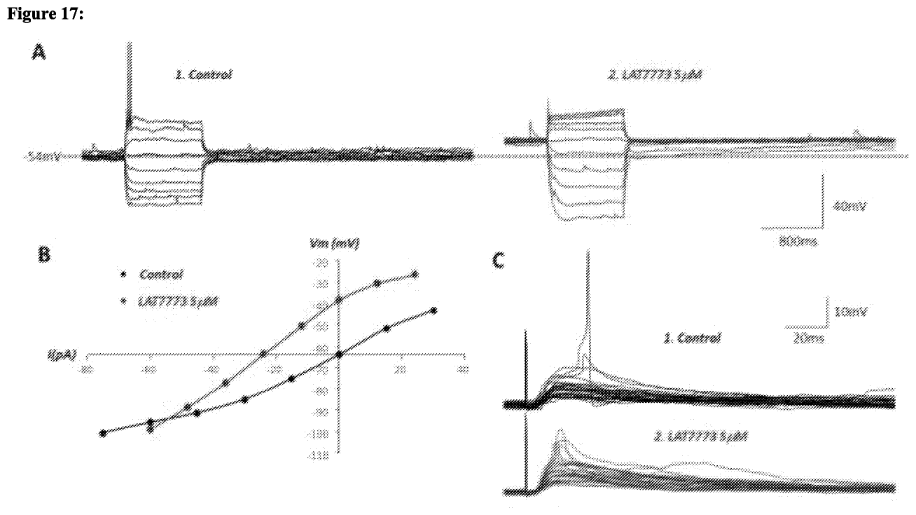

We closer inspected those cells where clear responses were observed. LAT7773-induced responses were associated with no clear reversal potential in 7 neurones indicating either no effect, simultaneous inhibition and activation of conductances or engagement of ion exchangers or pumps. In one neurone, LAT7773 induced excitation with an increase in neuronal input resistance and reversal potential around −93 mV, close to the reversal potential for potassium ions (FIG. 17). This suggest excitation induced by LAT7773 in this cell was mediated via block of one or more potassium conductances. In another cell, little change in input resistance was observed in LAT7773 around the resting potential but inward rectification observed as a decrease in the slope of W relations beyond potentials more negative than around −75 mV was enhanced (FIG. 16). Generally other than two cells, the ionic mechanisms underlying LAT7773-induced postsynaptic effects were unclear.

### (ii) The Effect of LAT 7773 on Dorsal Root Afferent-Mediated Synaptic Inputs

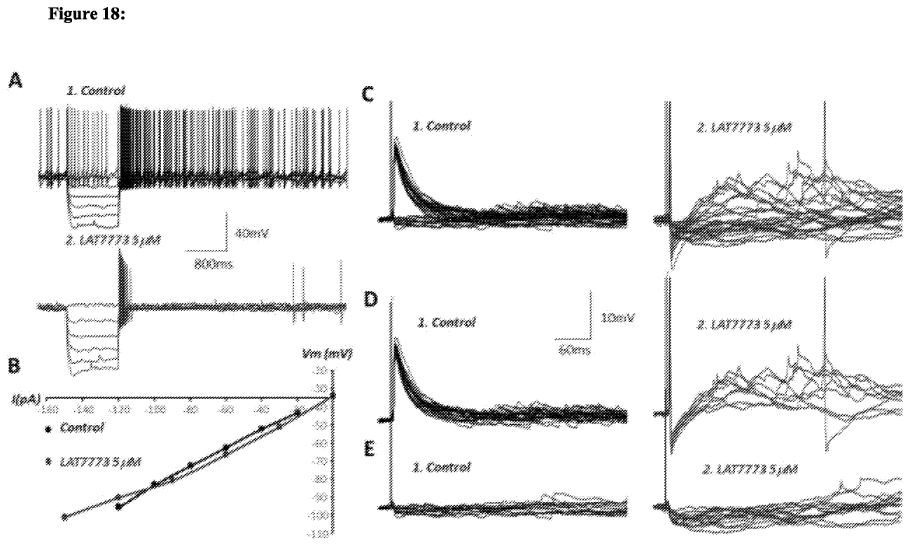

The effects of LAT7773 were also tested on dorsal root afferent-mediated synaptic inputs. LAT7773 had similar variable effects on synaptic inputs. In three neurones electrical stimulation of dorsal roots (0.1 Hz) evoked EPSPs that were suppressed in LAT7773 (see FIG. 16). In two other neurones EPSPs, and in one of these IPSPs, were enhanced in LAT7773 whereas the remaining two neurones showed no change in the presence of the compound (see FIGS. 17 and 18).

TABLE 3
Summary data of changes in membrane potential and input resistance
associated with LAT7773-induced responses in dorsal horn
neurones from Chung models of neuropathic pain.

| | Rest | LAT7773 5 μM | ΔVm |
|---|---|---|---|
| | Membrane Potential (mV) | | |
| Mean | −57.2 | −50.3 | −6.9 |
| SEM | 2.3 | 3.0 | 3.0 |
| n | 9 | 9 | 9 |
| P | | 0.050 | |
| | Normalised Membrane Potential | | |
| Mean | 100 | 88.4 | 11.6 |
| SEM | | 5.0 | 5.0 |
| n | 9 | 9 | 9 |
| P | | 0.048 | |
| | Control | LAT7773 5 μM | ΔIR |
| | Input Resistance (MΩ) | | |
| Mean | 350.6 | 373.5 | −22.9 |
| SEM | 68.1 | 103.6 | 45.1 |
| n | 9 | 9 | 9 |
| P | | 0.625 | |
| | Normalised Input Resistance | | |
| Mean | 100 | 99.9 | 0.1 |
| SEM | | 12.5 | 12.5 |
| n | 9 | 9 | 9 |
| P | | 0.993 | |

### Example 4

### The Effects of LAT7774 on Dorsal Horn Neurones

### (i) Effect of LAT 7774 on Membrane Properties of Dorsal Horn Neurones

The effects of LAT7774 (5 μM) were investigated on 7 dorsal horn neurones in spinal cord slices prepared from Chung models of neuropathic pain, recorded ipsilateral to the site of injury. In these neurones, LAT7774 induced a marked membrane hyperpolarisation and inhibition of activity in 2 neurones, ranging between 9.0 and 25.2 mV, a marked membrane depolarisation in 3 neurones ranging between 5.4 and 25.1 mV and had little effect on a further 2 dorsal horn neurones.

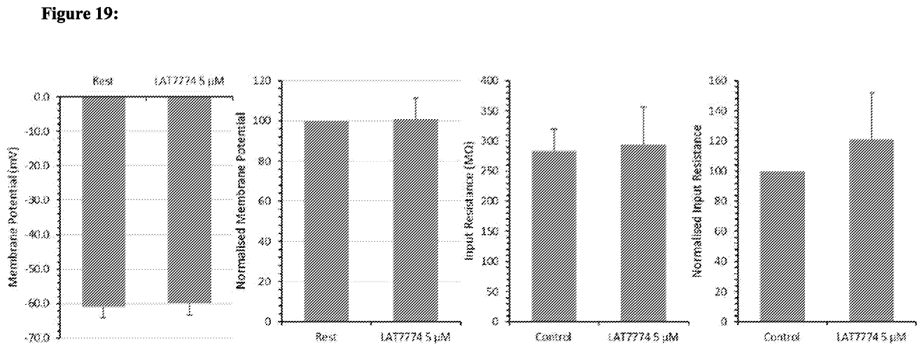

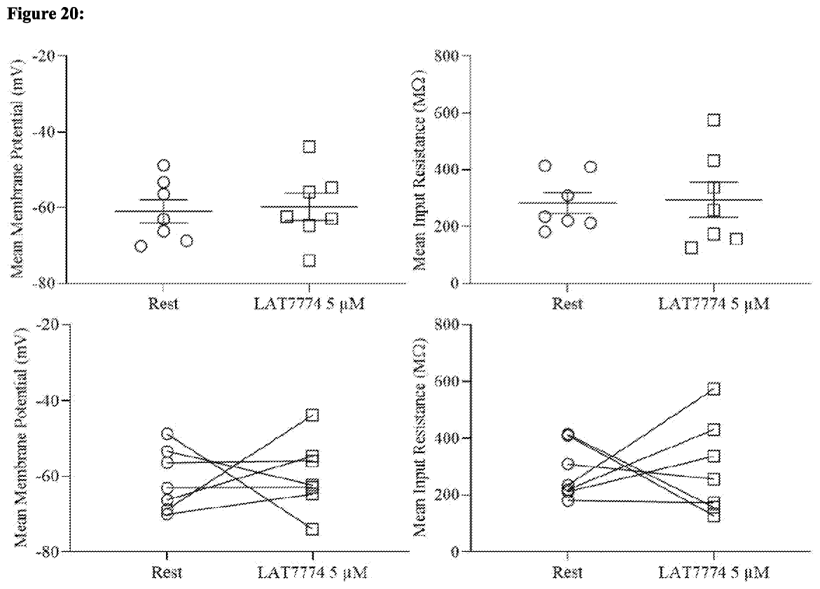

Data for all cells (n=9) showed LAT7774 induced a modest membrane depolarisation from a mean resting membrane potential of −61.0±3.1 mV to a new steady-state resting potential of −59.8±3.6 mV, amounting to a 1.2±6.0 mV change in membrane potential (n=7, P=0.848, Table 4). LAT7774-induced responses were associated overall with an increase in neuronal input resistance from a mean control resting level of 283.2±36.6 MΩ to 294.0±62.2 MΩ (n=7, P=0.907) in the presence of LAT7774, amounting to a 10.8±88.1 MΩ increase in neuronal input resistance. FIGS. 19 and 20 summarises the effects of LAT7774 on membrane properties of dorsal horn neurones (see also Table 4).

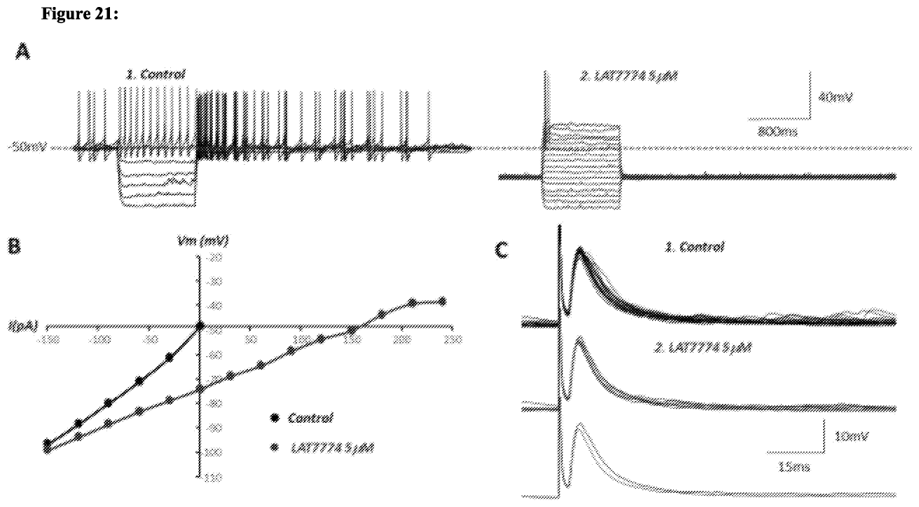

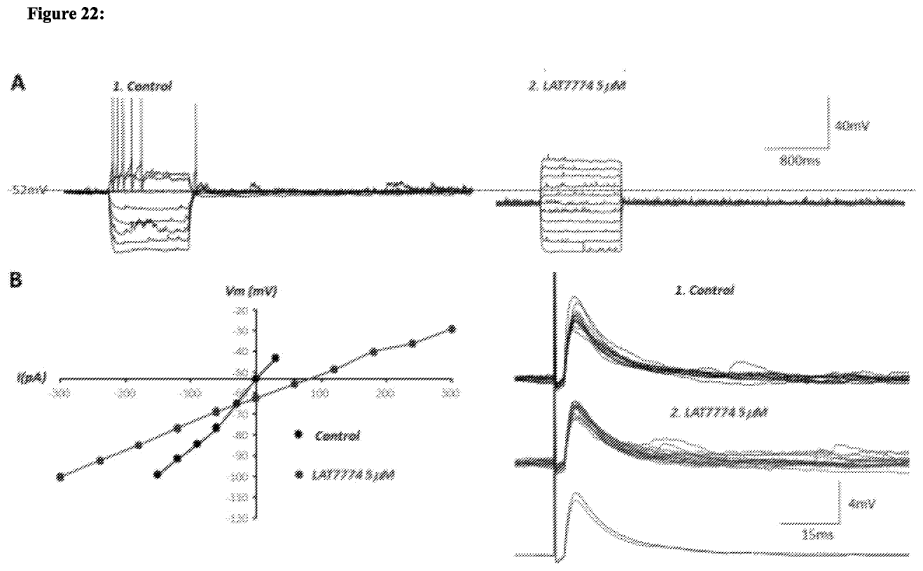

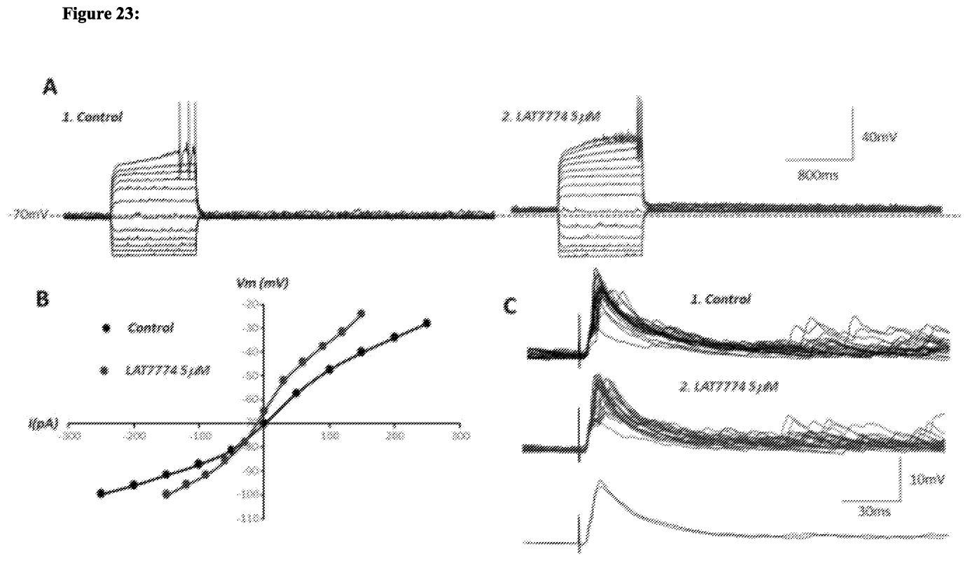

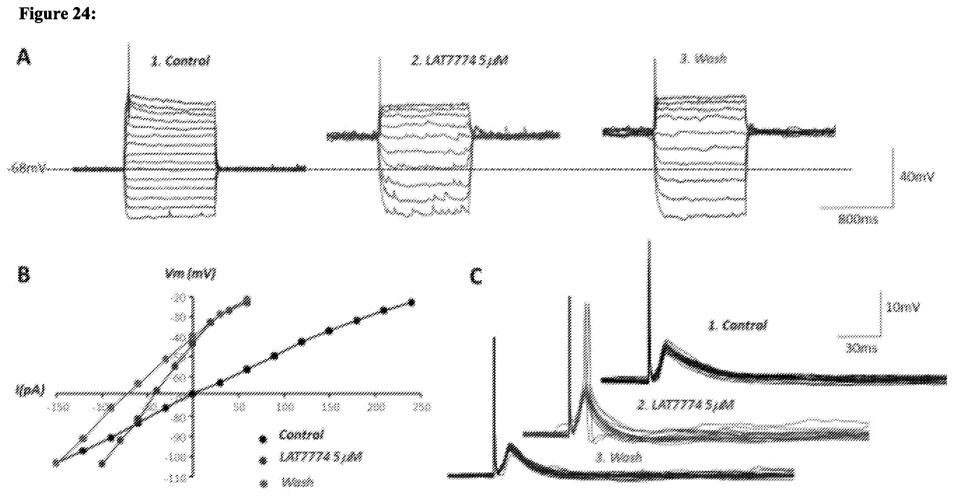

We closer inspected those cells where clear responses were observed. In two neurones, LAT7774 induced a clear membrane hyperpolarisation associated with a reduction in neuronal input resistance with reversal potentials around −100 mV in one cell and −65 mV in another, suggesting activation of a potassium conductance and chloride conductance, respectively (see FIGS. 21 and 22). In three other neurones LAT7774 induced membrane depolarisation associated with an increase in neuronal input resistance and reversal potentials around −80 mV, −80 mV and −85 mV, respectively, all close to the reversal potential for potassium ions under our recording conditions (see FIGS. 23 and 24). These data suggest LAT7774 induced depolarisation via block of one or more resting potassium conductances. Furthermore, in one cell, membrane depolarisation was also associated with changes in inward rectification suggesting part of the action of this compound may be to block or change the voltage sensitivity of inwardly rectifying potassium conductances (see FIG. 23).

### (ii) The Effect of LAT 774 on Dorsal Root Afferent-Mediated Synaptic Inputs

The effects of LAT7774 were also tested on dorsal root afferent-mediated synaptic inputs. LAT7774 had no effect on dorsal root evoked (0.1 Hz) EPSPs in 4 neurones, induced a minor reduction in two and increased EPSPs in one driving some EPSPs to reach threshold for firing (see FIGS. 22 to 25).

TABLE 4
Summary data of changes in membrane potential and input resistance
associated with LAT7774-induced responses in dorsal horn
neurones from Chung models of neuropathic pain.

| | Rest | LAT7774 5 μM | ΔVm |
|---|---|---|---|
| | Membrane Potential (mV) | | |
| Mean | −61.0 | −59.8 | −1.2 |
| SEM | 3.1 | 3.6 | 6.0 |
| n | 7 | 7 | 7 |
| P | | 0.848 | |
| | Normalised Membrane Potential | | |
| Mean | 100 | 100.9 | −0.9 |
| SEM | | 10.5 | 10.5 |
| n | 7 | 7 | 7 |
| P | | 0.937 | |
| | Control | LAT7774 5 μM | ΔIR |
| | Input Resistance (MΩ) | | |
| Mean | 283.2 | 294.0 | −10.8 |
| SEM | 36.6 | 62.2 | 88.1 |
| n | 7 | 7 | 7 |
| P | | 0.907 | |
| | Normalised Input Resistance | | |
| Mean | 100 | 121.2 | −21.2 |
| SEM | | 30.8 | 30.8 |
| n | 7 | 7 | 7 |
| P | | 0.517 | |

### Example 5

### The Effects of prolactin on Dorsal Horn Neurones

(i) Effect of prolactin on Membrane Properties of Dorsal Horn Neurones

The effects of prolactin (500 nM) were investigated on 3 dorsal horn neurones in spinal cord slices prepared from Chung models of neuropathic pain, recorded ipsilateral to the site of injury. In these neurones, prolactin induced a small membrane depolarisation in 2 neurones and had little effect on a further dorsal horn neurone.

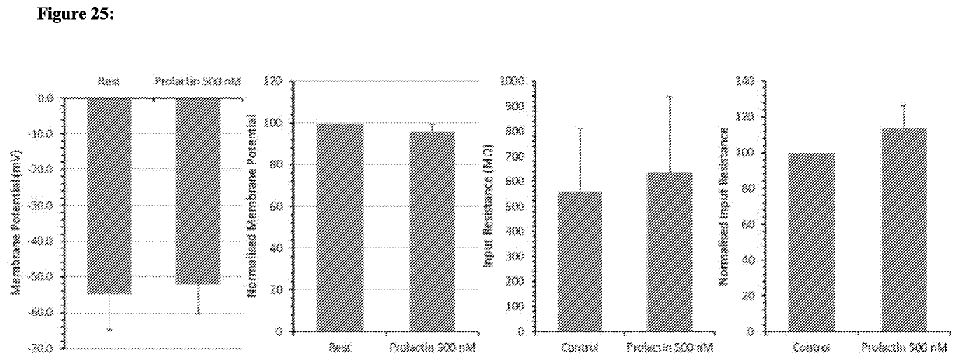

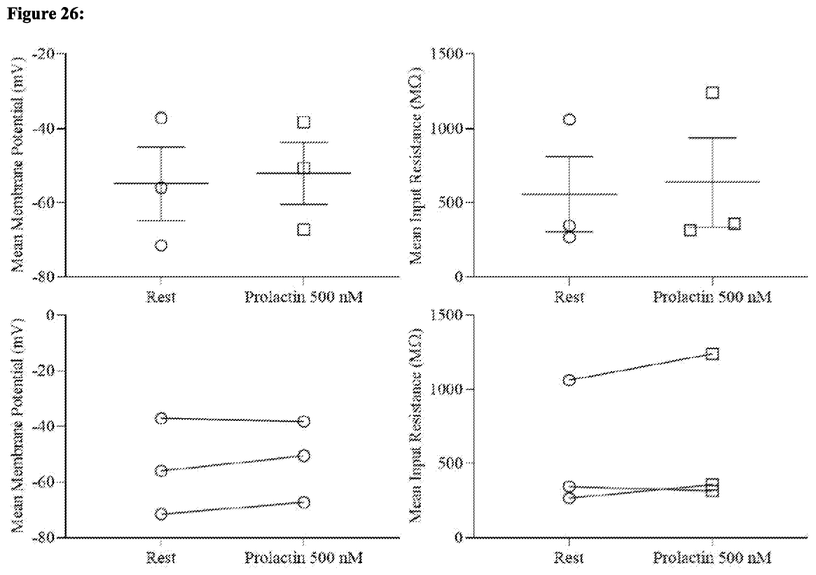

Data for all cells (n=3) showed prolactin induced a marginal membrane depolarisation from a mean resting membrane potential of −54.9±10.0 mV to a new steady-state resting potential of −52.0±8.4 mV, amounting to a 2.8±2.0 mV change in membrane potential (n=3, P=0.291, Table 5). Prolactin-induced responses were associated overall with an increase in neuronal input resistance from a mean control resting level of 558.3±252.9 MS2 to 637.3±301.1 MS2 (n=3, P=0.322) in the presence of prolactin, amounting to a 79.0±60.5 MS2 increase in neuronal input resistance. FIGS. 25 and 26 summarises the effects of prolactin on membrane properties of dorsal horn neurones to-date (see also Table 5).

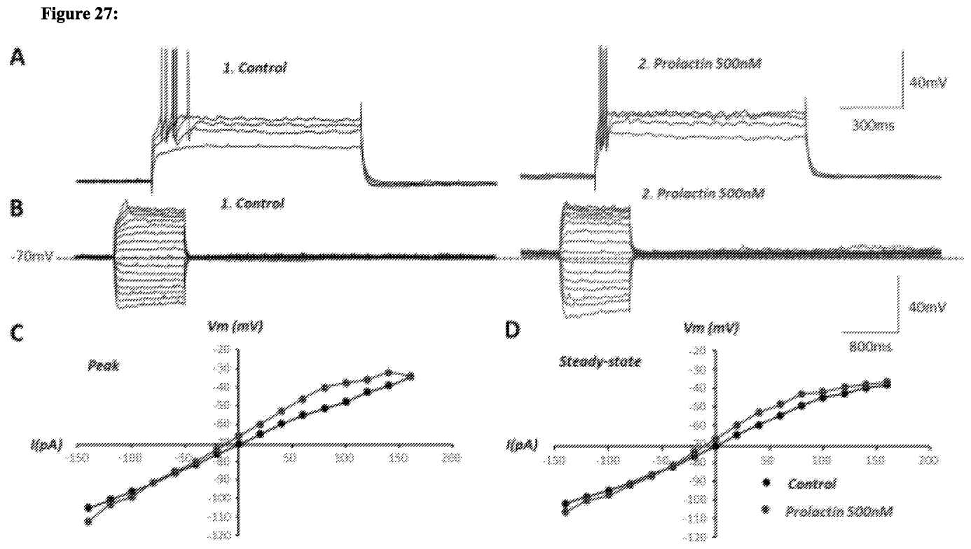

We closer inspected the effects of prolactin on these cells. In two neurones prolactin had very little effect. However, in one cell a clear membrane depolarisation was noted associated with an increase in neuronal input resistance. From VI relations the reversal potential of this prolactin-induced depolarisation was around −90 mV, close to the reversal potential for potassium ions under our recording conditions (see FIG. 27). These data suggest prolactin induced depolarisation by suppressing one or more resting potassium conductances.

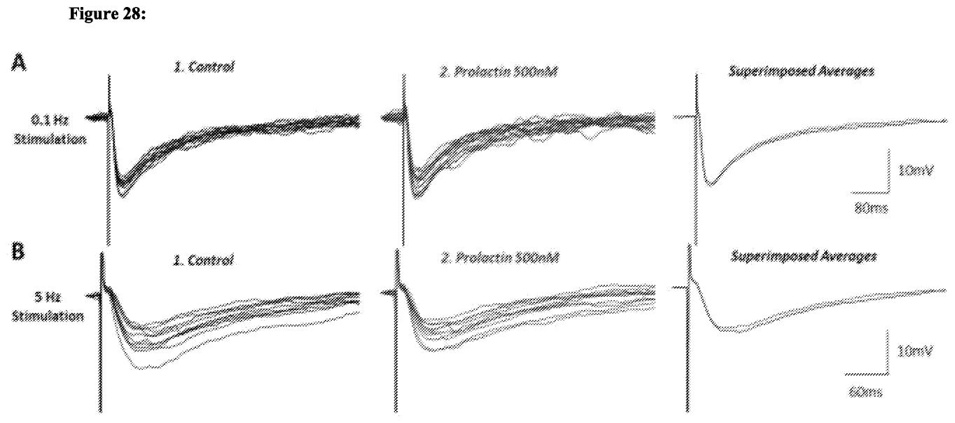

(ii) The Effect of prolactin on Dorsal Root Afferent-Mediated Synaptic Inputs

Wherever possible, the effects of prolactin were also tested on dorsal root afferent-mediated synaptic inputs. Prolactin had no effect on IPSPs evoked by dorsal root stimulation in one cell (FIG. 28).

TABLE 5
Summary data of changes in membrane potential and input resistance
associated with prolactin-induced responses in dorsal horn
neurones from Chung models of neuropathic pain.

| | Rest | Prolactin 500 nM | ΔVm |
|---|---|---|---|
| | Membrane Potential (mV) | | |
| Mean | −54.9 | −52.0 | −2.8 |
| SEM | 10.0 | 8.4 | 2.0 |
| n | 3 | 3 | 3 |
| P | | 0.291 | |
| | Normalised Membrane Potential | | |
| Mean | 100 | 95.8 | 4.2 |
| SEM | | 3.7 | 3.7 |
| n | 3 | 3 | 3 |
| P | | 0.378 | |

| | Control | Prolactin 500 nM | ΔIR |
|---|---|---|---|
| | Input Resistance (MΩ) | | |
| Mean | 558.3 | 637.3 | −79.0 |
| SEM | 252.9 | 301.1 | 60.5 |
| n | 3 | 3 | 3 |
| P | | 0.322 | |
| | Normalised Input Resistance | | |
| Mean | 100 | 113.9 | −13.9 |
| SEM | | 12.6 | 12.6 |
| n | 3 | 3 | 3 |
| P | | 0.384 | |

### Example 6

### In Vivo Study to Compare the Efficacy of Oral and Intramuscular Administration of LAT8881 and LAT7771 on Paw Withdrawal Threshold in a Rat Model of Neuropathic Pain

Chung model of neuropathic pain was used to compare the in vivo efficacy of LAT8881 and LAT7771 administered orally or intramuscularly.

The spinal nerve ligation model (Chung model) was first reported by Kim and Chung (1992; Pain, 50(3):355-63) and involves a single tight ligation of the L5 spinal nerve. The model shows characteristic features of neuropathic pain symptoms/signs such as: mechanical allodynia, mechanical and thermal hyperalgesia and spontaneous pain that mimics the symptoms/signs observed in clinical patients. This model has been used as a “gold standard' model for assessing the efficacy of novel compounds targeting neuropathic pain.

Adult male Sprague-Dawley rats, 8-9 weeks old, weighing 220-250 g at the time of surgery, were anaesthetised with 3% isoflurane mixed with oxygen followed by an intramuscular injection of a mixture of ketamine and xylazine. The back was shaved and sterilised with povidone-iodine soaked cotton balls. The L5 spinal nerve was carefully isolated and tightly located with 6/0 silk suture and the wound was closed in layers. The single-dose antibiotics (Amoxipen, 100 mg/kg, i.p.) was routinely given for prevention of infection after surgery.

The animals were placed in individual Perspex boxes on a raised metal mesh for at least 40 minutes. Starting with the filament of lowest force (1 g), each filament was applied perpendicularly to the centre of the ventral surface of the paw until slightly bent for 6 seconds. If the animal withdrew or lifted its paw upon stimulation, a von Frey hair with force immediately lower than that tested was subsequently used. If no response was observed, then a hair with force immediately higher was tested. The lowest amount of force required to induce reliable responses (positive in 2 out of 3 trials) was recorded as the value of pain withdrawl threshold (PWT).

PWT was assessed once daily for three days before surgery and once a week for monitoring the development of mechanical allodynia.

All drug tests were carried out on the 13^{th} to 17^{th} day after surgery. All drug dosing was ‘blindly’ carried out by a second experimenter. The test compounds were administered and PWT was assessed before and 2 hours following drug or vehicle (5% DMSO) administration.

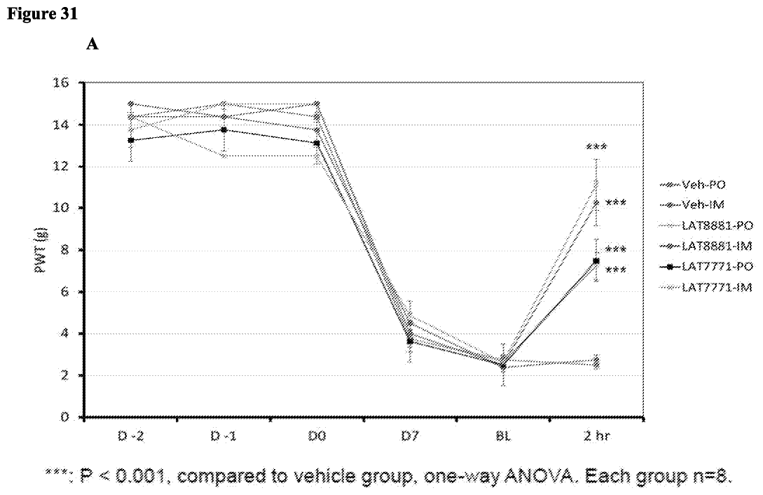

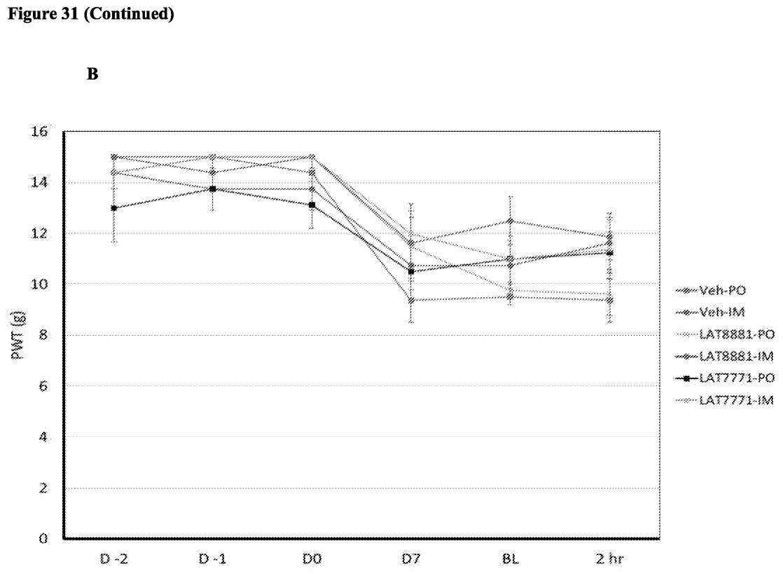

LAT7771 and LAT8881 improved the outcome on neuropathic pain, as indicated by the reversal of mechanical allodynia in Chung model of neuropathic pain. PWT was assessed daily for three days before surgery and once a week after surgery for monitoring the development of mechanical allodynia. PWT was assessed before and 2 hours following drug or vehicle administration. LAT8881, at 10 mg/kg, administered by oral or intramuscular routes, reversed mechanical allodynia at quickly as two hours after administration of the drug. Similarly, LAT7771, at 10 mg/kg, administered by oral or intramuscular routes, significantly increased the PWT in Chung model rats. Comparison of effects of LAT8881 and LAT7771, via two administration routes (orally (PO) or intramuscularly (IM)) on PWT in Chung model rats, on the ipsilateral and contralateral side of the injury is represented in FIG. 31.

### Discussion

The above examples show that LAT7771, LAT7772, LAT7773 and LAT7774 (SEQ ID NO:2, 3, 4, and 5) are capable of treating and preventing pain. A summary of the effects of LAT7771, LAT7772, LAT7773 and LAT7774 in comparison to prolactin on dorsal horn neurones is shown in FIGS. 29 and 30.

LAT7771 principally induced membrane depolarisation of dorsal horn neurones with an associated marginal increase in neuronal input resistance. One of the most interesting features of LAT7771 relates to its effects on dorsal root afferent-mediated synaptic inputs. LAT7771 consistently reduced dorsal root-evoked EPSPs and in two neurones promoted inhibitory synaptic transmission. There are features of the properties of this compound that are similar to the human growth hormone derived compound LAT8881 of PCT/AU2019/050020, which has been shown to have analgesic properties, the effect of LAT7771 on synaptic transmission suggest significant potential to produce analgesia.

LAT7772 induced both membrane depolarisations and hyperpolarisations with the majority of responses being associated with a reduction in neuronal input resistance. The predominant effect of this compound on dorsal root-evoked synaptic inputs was also a suppressive effect on EPSPs. However, given the significant effect of this compound on neuronal input resistance it is unclear if these effects on dorsal root afferent-mediated excitatory inputs are direct or indirect as a result of changes to the postsynaptic cell.

LAT7773 principally induced membrane depolarisation of dorsal horn neurones with an associated increase in neuronal input resistance. Similarly, dorsal root afferent-mediated excitatory synaptic inputs were in some neurones depressed whilst in others they were clearly potentiated, the latter suggesting a tendency to be pro-analgesic.

The effects of LAT7774 were variable inducing both membrane depolarization. The effects of this compound were more variable on dorsal root afferent-mediated synaptic inputs, both facilitating and inhibiting these inputs.

Prolactin principally induced membrane depolarisation of dorsal horn neurones with an associated increase in neuronal input resistance consistent with block of a potassium conductance.

LAT7771 has shown analgesic properties in vivo comparable to that of LAT8881.

TABLE 6
SEQ ID NOs and corresponding amino acid sequences

| | |
|---|---|
| SEQ ID NO: 1 | R¹-C-R-I-X₁-X₂-X₃-X₄-N-C-R² |
| SEQ ID NO: 2 | CRIIHNNNC |
| SEQ ID NO: 3 | CRIIHNNNCG |
| SEQ ID NO: 4 | CRIVYDSNC |
| SEQ ID NO: 5 | CRIVYDSNCG |
| SEQ ID NO: 6 | YLKLLKCRIIHNNNC |
| SEQ ID NO: 7 | LKLLKCRIIHNNNC |
| SEQ ID NO: 8 | KLLKCRIIHNNNC |
| SEQ ID NO: 9 | LLKCRIIHNNNC |
| SEQ ID NO: 10 | LKCRIIHNNNC |
| SEQ ID NO: 11 | KCRIIHNNNC |
| SEQ ID NO: 12 | YLKLLKCRIIHNNNCG |
| SEQ ID NO: 13 | LKLLKCRIIHNNNCG |
| SEQ ID NO: 14 | KLLKCRIIHNNNCG |
| SEQ ID NO: 15 | LLKCRIIHNNNCG |
| SEQ ID NO: 16 | LKCRIIHNNNCG |
| SEQ ID NO: 17 | KCRIIHNNNCG |
| SEQ ID NO: 18 | YLKLLKCRIVYDSNC |
| SEQ ID NO: 19 | LKLLKCRIVYDSNC |
| SEQ ID NO: 20 | KLLKCRIVYDSNC |
| SEQ ID NO: 21 | LLKCRIVYDSNC |
| SEQ ID NO: 22 | LKCRIVYDSNC |
| SEQ ID NO: 23 | KCRIVYDSNC |
| SEQ ID NO: 24 | YLKLLKCRIVYDSNCG |
| SEQ ID NO: 25 | LKLLKCRIVYDSNCG |
| SEQ ID NO: 26 | KLLKCRIVYDSNCG |
| SEQ ID NO: 27 | LLKCRIVYDSNCG |
| SEQ ID NO: 28 | LKCRIVYDSNCG |
| SEQ ID NO: 29 | KCRIVYDSNCG |
| SEQ ID NO: 30 | MNIKGSPWKGSLLLLLVSNLLLCQSVAPLPICPGGAARCQ |
| | VTLRDLFDRAVVL |
| | SHYIHNLSSEMFSEFDKRYTHGRGFITKAINSCHTSSLATP |
| | EDKEQAQQMNQK |
| | DFLSLIVSILRSWNEPLYHLVTEVRGMQEAPEAILSKAVEI |
| | EEQTKRLLEGMEL |
| | IVSQVHPETKENEIYPVWSGLPSLQMADEESRLSAYYNLL |
| | HCLRRDSHKIDNY |
| | LKLLKCRIIHNNNC |

The claims defining the invention are as follows:

1. A method of treating or preventing neuropathic pain in a subject, the method comprising administering to a subject a therapeutically effective amount of prolactin, or a functional variant thereof, wherein the functional variant comprises a peptide of formula (I) or a pharmaceutically acceptable salt thereof:
(I)
(SEQ ID NO: 1)
R¹-C-R-I-X₁-X₂-X₃-X₄-N-C-R²
wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent.

2. The method of claim 1, wherein the functional variant is selected from the group consisting of CRIIHNNNC (SEQ ID NO:2), CRIIHNNNCG (SEQ ID NO:3), CRIVYDSNC (SEQ ID NO:4) and CRIVYDSNCG (SEQ ID NO:5).

3. The method of claim 2, wherein the functional variant is CRIIHNNNC (SEQ ID NO:2).

4. The method of claim 2, wherein the functional variant is CRIIHNNNCG (SEQ ID NO:3).

5. The method of claim 2, wherein the functional variant is CRIVYDSNC (SEQ ID NO:4).

6. The method of claim 2, wherein the functional variant is CRIVYDSNCG (SEQ ID NO:5).

7. The method of anyone of claims 1 to 6, wherein said therapeutically effective amount alleviates neuropathic pain in the subject.

8. The method of any one of claims 1 to 7, wherein the subject is a human.

9. The method of any one of claims 1 to 8, wherein the neuropathic pain is associated with a condition selected from the group consisting of diabetic neuropathy; Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HIV sensory neuropathy; hereditary motor and sensory neuropathy (HMSN); hereditary sensory neuropathy (HSN); hereditary sensory and autonomic neuropathy; hereditary neuropathy with ulcero-mutilation; nitrofurantoin neuropathy; tomaculous neuropathy; neuropathy caused by nutritional deficiency, neuropathy caused by kidney failure, trigeminal neuropathic pain, atypical odontalgia (phantom tooth pain), burning mouth syndrome, complex regional pain syndrome, repetitive strain injury, drug-induced peripheral neuropathy and peripheral neuropathy associated with infection.

10. The method of any one of claims 1 to 9, further comprising administering to the subject a therapeutically effective amount of a second agent capable of alleviating pain in the subject, wherein the second agent is not prolactin, or a functional variant thereof according to any one of claims 1 to 10.

11. The method of claim 10, wherein the second agent is capable of alleviating neuropathic pain in the subject.

12. The method of claim 10, wherein the second agent is capable of alleviating nociceptive pain in the subject.

13. A pharmaceutical composition comprising prolactin, or a functional variant thereof, for use in the treatment or prevention of neuropathic pain in a subject, wherein the functional variant comprises a peptide of formula (I):
(I)
(SEQ ID NO: 1)
R¹-C-R-I-X₁-X₂-X₃-X₄-N-C-R²
wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent.

14. The composition for use according to claim 13, wherein the functional variant is selected from the group consisting of CRIIHNNNC (SEQ ID NO:2), CRIIHNNNCG (SEQ ID NO:3), CRIVYDSNC (SEQ ID NO:4) and CRIVYDSNCG (SEQ ID NO:5).

15. The composition for use according to claim 14, wherein the functional variant is
(SEQ ID NO: 2)
CRIIHNNNC.

16. The composition for use according to claim 14, wherein the functional variant is
(SEQ ID NO: 3)
CRIIHNNNCG.

17. The composition for use according to claim 14, wherein the functional variant is
(SEQ ID NO: 4)
CRIVYDSNC.

18. The composition for use according to claim 14, wherein the functional variant is
(SEQ ID NO: 5)
CRIVYDSNCG.

19. The composition for use according to any one of claims 13 to 18, wherein the prolactin, or functional variant thereof, is present in a therapeutically effective amount that, when administered to a subject, alleviates neuropathic pain in the subject in the absence of a therapeutically effective analgesic effect on nociceptive pain.

20. The composition for use according to any one of claims 13 to 19, wherein the subject is a human.

21. The composition for use according to any one of claims 13 to 19, wherein the neuropathic pain is associated with a condition selected from the group consisting of diabetic neuropathy; Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HW sensory neuropathy; hereditary motor and sensory neuropathy (HMSN); hereditary sensory neuropathy (HSN); hereditary sensory and autonomic neuropathy; hereditary neuropathy with ulcero-mutilation; nitrofurantoin neuropathy; tomaculous neuropathy; neuropathy caused by nutritional deficiency, neuropathy caused by kidney failure, trigeminal neuropathic pain, atypical odontalgia (phantom tooth pain), burning mouth syndrome, complex regional pain syndrome, repetitive strain injury, drug-induced peripheral neuropathy and peripheral neuropathy associated with infection.

22. The composition for use according to any one of claims 13 to 21, further comprising a second agent capable of alleviating pain in the subject, wherein the second agent is not prolactin, or a functional variant thereof.

23. The composition for use according to claim 22, wherein the second agent is capable of alleviating neuropathic pain in the subject.

24. The composition for use according to claim 22, wherein the second agent is capable of alleviating nociceptive pain in the subject.

25. Use of prolactin, or a functional variant thereof, in the manufacture of a medicament for the treatment or prevention of neuropathic pain in a subject, wherein the functional variant comprises a peptide of formula (I):
(I)
(SEQ ID NO: 1)
R¹-C-R-I-X₁-X₂-X₃-X₄-N-C-R²
wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent.

26. Use of claim 25, wherein the functional variant is selected from the group consisting
(SEQ ID NO: 2)
of CRIIHNNNC,
(SEQ ID NO: 3)
CRIIHNNNCG,
(SEQ ID NO: 4)
CRIVYDSNC
and
(SEQ ID NO: 5)
CRIVYDSNCG.

27. Use of claim 26, wherein the functional variant is CRIIHNNNC (SEQ ID NO:2)

28. Use of claim 26, wherein the functional variant is CRIIHNNNCG (SEQ ID NO:3)

29. Use of claim 26, wherein the functional variant is CRIVYDSNC (SEQ ID NO:4).

30. Use of claim 26, wherein the functional variant is CRIVYDSNCG (SEQ ID NO:5).

31. Use of any one of claims 25 to 30, wherein the prolactin, or functional variant thereof, is formulated for administration to the subject in a therapeutically effective amount that alleviates neuropathic pain in the subject in the absence of a therapeutically effective analgesic effect on nociceptive pain.

32. Use of any one of claims 25 to 31, wherein the subject is a human.

33. Use of any one of claims 25 to 32, wherein the neuropathic pain is associated with a condition selected from the group consisting of diabetic neuropathy; Herpes Zoster (shingles)-related neuropathy; fibromyalgia; multiple sclerosis, stroke, spinal cord injury; chronic post-surgical pain, phantom limb pain, Parkinson's disease; uremia-associated neuropathy; amyloidosis neuropathy; HW sensory neuropathy; hereditary motor and sensory neuropathy (HMSN); hereditary sensory neuropathy (HSN); hereditary sensory and autonomic neuropathy; hereditary neuropathy with ulcero-mutilation; nitrofurantoin neuropathy; tomaculous neuropathy; neuropathy caused by nutritional deficiency, neuropathy caused by kidney failure, trigeminal neuropathic pain, atypical odontalgia (phantom tooth pain), burning mouth syndrome, complex regional pain syndrome, repetitive strain injury, drug-induced peripheral neuropathy and peripheral neuropathy associated with infection.

34. Use of any one of claims 25 to 33, wherein the prolactin, or functional variant thereof, is formulated for administration sequentially, or in combination, with a second agent capable of alleviating pain in the subject, wherein the second agent is not prolactin, or a functional variant thereof.

35. Use of claim 34, wherein the second agent is capable of alleviating neuropathic pain in the subject.

36. Use of claim 35, wherein the second agent is capable of alleviating nociceptive pain in the subject.

37. A composition comprising a therapeutically effective amount of prolactin-derived peptide consisting of amino acid sequence CRIIHNNNC (SEQ ID NO:2) or amino acid sequence CRIIHNNNCG (SEQ ID NO:3) or amino acid sequence CRIVYDSNC (SEQ ID NO:4) or amino acid sequence CRIVYDSNCG (SEQ ID NO:5).

38. The composition of claim 37, further comprising a pharmaceutically acceptable carrier, excipient or diluent.

39. The composition of claim 37 or claim 38, formulated for oral administration.

40. A composition comprising a peptide of SEQ ID NO: 2 or SEQ ID NO:3 or SEQ ID NO: 4 or SEQ ID NO:5, or a pharmaceutically acceptable salt of any of the foregoing, for use as a medicament.

41. A pharmaceutical composition comprising: (i) prolactin, or a functional fragment thereof, wherein the functional fragment comprised a peptide of formula (I):
(I)
(SEQ ID NO: 1)
R¹-C-R-I-X₁-X₂-X₃-X₄-N-C-R²
wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent, and
(ii) a second agent capable of alleviating pain in the subject, wherein the second agent is not prolactin, or a functional variant thereof.

42. The composition of claim 41, wherein the functional variant is selected from the group consisting of CRIIHNNNC (SEQ ID NO:2), CRIIHNNNCG (SEQ ID NO:3), CRIVYDSNC (SEQ ID NO:4) and CRIVYDSNCG (SEQ ID NO:5).

43. The composition of claim 42, wherein the functional variant is CRIIHNNNC (SEQ ID NO:2).

44. The composition of claim 42, wherein the functional variant is CRIIHNNNCG (SEQ ID NO:3).

45. The composition of claim 42, wherein the functional variant is CRIVYDSNC (SEQ ID NO:4)

46. The composition of claim 42, wherein the functional variant is CRIVYDSNCG (SEQ ID NO:5).

47. The composition of any one of claims 41 to 46, wherein the second agent is capable of alleviating neuropathic pain in the subject.

48. The composition of any one of claims 41 to 46, wherein the second agent is capable of alleviating nociceptive pain in the subject.

49. An analgesic composition comprising prolactin, or a functional variant thereof, wherein the functional variant comprises a peptide of formula (I):
(I)
(SEQ ID NO: 1)
R¹-C-R-I-X₁-X₂-X₃-X₄-N-C-R²
wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent.

50. The composition of claim 49, wherein the functional variant is selected from the group consisting of CRIIHNNNC (SEQ ID NO:2), CRIIHNNNCG (SEQ ID NO:3), CRIVYDSNC (SEQ ID NO:4) and CRIVYDSNCG (SEQ ID NO:5).

51. The composition of claim 50, wherein the functional variant is CRIIHNNNC (SEQ ID NO:2).

52. The composition of claim 50, wherein the functional variant is CRIIHNNNCG (SEQ ID NO:3).

53. The composition of claim 50, wherein the functional variant is CRIVYDSNC (SEQ ID NO:4)

54. The composition of claim 50, wherein the functional variant is CRIVYDSNCG (SEQ ID NO:5).

55. The composition of any one of claims 49 to 54, further comprising a second agent capable of alleviating pain in the subject, wherein the second agent is not prolactin, or a functional variant thereof according to any one of claims 1 to 54.

56. The composition of claim 55, wherein the second agent is capable of alleviating neuropathic pain in the subject.

57. The composition of claim 55, wherein the second agent is capable of alleviating nociceptive pain in the subject.

58. An analgesic composition comprising a therapeutically effective amount of prolactin, or a functional variant thereof, wherein the functional variant consists of amino acid sequence CRIIHNNNC (SEQ ID NO:2) or amino acid sequence CRIIHNNNCG (SEQ ID NO:3) or amino acid sequence CRIVYDSNC (SEQ ID NO:4) or amino acid sequence CRIVYDSNCG (SEQ ID NO:5).

59. The composition of claim 58, further comprising a pharmaceutically acceptable carrier, excipient or diluent.

60. The composition of claim 58 or claim 59, formulated for oral administration.

61. An analgesic composition comprising a peptide selected from the group consisting of any one of SEQ ID NOs: 2 to 5, or a pharmaceutically acceptable salt thereof, for use as a medicament.

62. A pharmaceutical composition comprising prolactin, or a functional variant thereof, wherein the functional variant comprises a peptide of formula (I):
(I)
(SEQ ID NO: 1)
R¹-C-R-I-X₁-X₂-X₃-X₄-N-C-R²
wherein
X₁ is an amino acid residue selected from isoleucine (I) and valine (V);
X₂ is an amino acid residue selected from histidine (H) and tyrosine (Y);
X₃ is an amino acid residue selected from aspartic acid (D) and asparagine (N);
X₄ is an amino acid residue selected from asparagine (N) and serine (S);
R¹ is selected from the group consisting of YLKLLK, LKLLK, KLLK, LLK, LL, K or R¹ is absent; and
R² is G (glycine), or R² is absent.
